(19) United States
(12) Patent Application Publication
(10) Pub. No.: US 2008/0206316 A1
(43) Pub. Date: Aug. 28, 2008

(54) Chromium-Fatty Acid Compounds and Methods of Making and Using Thereof

(76) Inventors: Colin Barrow, Halifax (CA); Jaroslav A. Kralovec, Halifax (CA); Harry Stephen Ewart, Halifax (CA)

Correspondence Address: NEEDLE & ROSENBERG, P.C., SUITE 1000, 999 PEACHTREE STREET, ATLANTA, GA, 30309-3915, US

(21) Appl. No.: 10/575,204

(22) PCT Filed: Jan. 26, 2006
(86) PCT No.: PCT/IB2006/001526

§ 371 Date: Apr. 10, 2008

Related U.S. Application Data
(60) Provisional application No. 60/647,703, filed on Jan. 27, 2005.

(51) Int. Cl. A61K 9/127; C07F 11/00; C07F 3/06; A61K 9/48; A61P 3/00; A61K 9/50; A61K 31/28

(52) U.S. Cl. 424/450; 554/71; 554/75; 514/505; 424/490; 424/451; 424/492

(57) ABSTRACT

Disclosed are compounds comprising one or more chromium atoms bonded to one or more fatty acids. Also disclosed are nutritional supplements, pharmaceutical formulations, delivery devices, and foodstuffs comprising the disclosed compounds. Methods of using the disclosed compounds and compositions to improve health are also disclosed.

### CROSS REFERENCE TO RELATED APPLICATIONS

This application claims the benefit of priority to U.S. Provisional Application No. 60/647,703, filed Jan. 27, 2005, which is incorporated by reference herein in its entirety.

### FIELD

The disclosed matter relates to compounds comprising chromium and a fatty acid, including methods of making and using such compounds.

### BACKGROUND

Essential trace elements are an important component of a healthy diet, and deficiencies of such elements can result in the impairment of various physiological functions. Chromium is one such essential trace element and has recently received much attention.

Chromium(III) is a cofactor for insulin action and plays a role in the peripheral activities of insulin by forming a ternary complex with insulin receptors. Biologically active chromium, also referred as glucose tolerance factor (GTF), is believed to be a dinicotinato-chromium(III) glutathione-like complex (Evans, et al., Biochem Biophys Res Commun 50:718-22, 1973). GTF is responsible for binding insulin to cell membrane insulin receptor sites (Mertz, Physiol Rev 4:163-239, 1969). A higher level of understanding chromium biochemistry came with Japanese work describing the isolation and characterization of a unique chromium oligopeptide named low-molecular-weight chromium-binding substance (LMWCr or chromomodulin) (Wada, et al., Environ Res 32:228-39, 1983). Chromomodulin is constructed of only four types of amino acid residues (G, C, D, E) and its apparent molecular weight is about 1500 Da. The material is widely distributed in mammals. This oligopeptide binds chromium(III) ions in response to insulin-mediated Cr(III) ion flux. The resulting metal complex then binds to insulin-stimulated insulin receptors and activates its tyrosine kinase activity. The chromomodulin also seems to play a role in the autoamplification of insulin signaling.

The recommended daily dietary intake of chromium for adults is from about 50 to about 200 μg (Marcus and Coulston, The Vitamins. In: The Pharmacological Basis of Therapeutics. Gilman, et al., eds., McGraw-Hill, Inc., New York, pp. 1524-7, 1990). A diet deficient in chromium has been found to lead to impaired glucose, lipid, and protein metabolism (Goyer, Toxic Effects of Metals. In: Casarett and Doulll's Toxicology. Amdur, et al., eds., 4th ed., Pergamon Press, New York, pp. 638-9, 1991). Further, chromium dietary deficiencies have been linked to both maturity-onset diabetes and to cardiovascular disease.

Dietary supplementation of chromium has been reported to lead to improvements in glucose tolerance, serum lipid concentrations, including high-density lipoprotein cholesterol, insulin and insulin binding (Anderson, Clin Psychol Biochem 4:31-41, 1986). For example, chromium compounds have been found to reduce blood glucose levels and are used to control certain cases of diabetes. Further, chromium compounds have been found to reduce blood cholesterol levels by diminishing the concentration of LDL in the blood. Supplemental chromium compounds have also been associated with improvements of risk factors associated with adult-onset (Type II) diabetes and cardiovascular disease.

While humans are capable of converting inactive chromium compounds into biologically active forms (ATSDR: Toxicological Profile for Chromium (ATSDR/TP-92/08), Atlanta, Ga., Agency for Toxic Substances and Disease Registry, p. 227, 1993), attempts to supplement dietary chromium by administering inorganic chromium compounds have not been particularly successful. Only about 0.5% of ingested inorganic chromium is assimilated into the body (Recommended Daily Allowances, Ninth Revised Edition, The National Academy of Sciences, p. 160, 1980). Wang, et al. showed that inorganic forms of Cr(III) were much less bio-available than organic coordination compounds (Nutr Res 9:989-98, 1989). Although inorganic chromium(III) and acetate salts show poor bio-availability, they exhibited limited activity as dietary supplements (Anderson and Kozlovsky, Am J Clin Nutr 41:1177-83, 1985). Chromium must be converted endogenously into an organic complex and must be consumed in a form of a biologically active molecule.

Various organic (simple or complex) chromium products have been prepared (Udy, Chromium. Vol. 1. Chemistry of Chromium and its Compounds. Reinhold Publ. Corp., New York, 1956). Chemical suppliers such as Aldrich list a number of inorganic salts and some organic salts including, chromium(III) acetate hydroxide [(CH₃COO)₇Cr₃(OH)₃], chromium(III) acetylacetonate, and chromium(III) benzoylacetonate. Chromic salts of other organic acids have been reported, including oxalates, formates, tartrates, glycollates, lactates, picrates, salicylates, etc. Out of the category of single chain fatty acids, the salts of C3-C9 entities were reported (Beilstein's Handbooch der Organischen Chemie, Vol. 4., Berlin, Springer, 1920). Viva Life Sciences, Inc. (Costa Mesa, Calif.) disclosed a technology to produce GTF-like activity chromium based on organochromium complexes containing nicotinate and glycine (U.S. Pat. No. 6,248,323 to Arnold, et al.).

One organic chromium complex that has achieved recent success as a dietary supplement is chromium picolinate, (tris-(2-pyridinecarboxylato-N¹,O²-chromium), a biologically active form of chromium. The compound was prepared early in the 1990's by Evans and Pouchnick by mixing CrCl₃ with bidentnate and picolinic acid (J Inorg Biochem 49:177-87, 1993). This compound is currently promoted as a muscle builder and weight-loss agent. Further, U.S. Pat. Nos. 5,087,623, 5,087,624, and 5,175,156, disclose the use of chromium picolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum HDL-cholesterol levels.

A physical mixture of chromium picolinate or nicotinate and a conjugated fatty acid or corresponding alcohol for treating insulin-dependent diabetes, improving insulin sensitivity, and reducing hyperlipidemia, including hypercholesterolemia, is disclosed in U.S. Pat. No. 6,809,115 to Katz, et al.

Catron discloses chromium(III) compounds with short chain acids containing from 3 to 7 carbon atoms (U.S. Pat. No. 5,846,581). The compounds are produced through a reaction that involves the short chain acids and sodium dichromate in the presence of a reducing agent such as glucose or propylene glycol. The products of the reaction are dark green, they solidify upon standing and are soluble in water. Chromium propionate, one of the described metal carboxylates, was found to be superior to chromium picolinate in effecting animal metabolism.

In light of the numerous health benefits associated with the essential trace element chromium, what is needed in the art are new compounds and compositions that can be used to supply chromium to subjects. Further, what it also needed are new methods of preparing and using such compounds and compositions. The compounds, compositions, and methods disclosed herein meet these needs.

### SUMMARY

In accordance with the purposes of the disclosed materials, compounds, compositions, articles, and methods, as embodied and broadly described herein, the disclosed subject matter, in one aspect, relates to compounds and compositions and methods for preparing and using such compounds and compositions. In another aspect, the disclosed subject matter relates to compounds comprising one or more chromium atoms bonded to one or more fatty acids, and to nutritional supplements, food stuffs, and pharmaceutical compositions comprising such compounds. In still another aspect, the disclosed subject matter relates to methods of preparing such chromium containing compounds and compositions. Still further, the disclosed subject matter relates to delivery devices containing such compounds and compositions and to methods of preparing the delivery devices. In yet another aspect, the disclosed subject matter relates to methods of using the described compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF FIGURES

The accompanying figures, which are incorporated in and constitute a part of this specification, illustrate several aspects described below.

FIG. 1 is a chemical structure of [Cr₃(H₂O)₃(μ-unsaturated fatty acid residue)₆(μ₃-O)]⁺.

FIG. 2 is an ESI mass spectrum of Cr-DHA complex dissolved in THF/methanol 90:10.

FIG. 3 is a comparison of isotope pattern for several multinuclear Cr complexes.

FIG. 4 is a MS/MS fragmentation spectrum of mass 2351.4 m/z.

FIG. 5 shows an intraperitoneal glucose tolerance test in non-diabetic and diabetic mice. Animals were fed a high fat/high sucrose diet with no added chromium for 2 weeks followed by daily inject of streptozotocin (35 mg/kg body weight) for 3 days. Animals were assessed prior to the streptozotocin injection protocol (non-diabetic) and 7 days following the start of the protocol (Diabetic). Results are the mean ±stand error of the mean of 5 or 6 mice per group.

FIG. 6 shows an intraperitoneal glucose tolerance test in diabetic mice fed the control and test diets. Animals were fed a high fat/high sucrose diet containing fish oil concentrate (1.5%) with no added chromium (Control) or with Cr-omega-3 conjugate (400 and 1000 μg elemental Cr/kg diet) or chromium picolinate (1000 μg elemental Cr/kg diet) for 4 weeks. Results are the mean ±stand error of the mean of 6 mice per group.

### DETAILED DESCRIPTION

The materials, compounds, compositions, articles, and methods described herein may be understood more readily by reference to the following detailed description of specific aspects of the disclosed subject matter and the Examples included therein and to the Figures.

Before the present materials, compounds, compositions, articles, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an unsaturated fatty acid” includes mixtures of two or more such unsaturated fatty acids, reference to “the microcapsule” includes mixtures of two or more such microcapsules, and the like.

“Optional” Or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

Reference will now be made in detail to specific aspects of the disclosed materials, compounds, compositions, articles, and methods, examples of which are illustrated in the accompanying Examples and Figures.

### Materials

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and a number of modifications that can be made to a number of components or residues of the compound are discussed, each and every combination and permutation that are possible are specifically contemplated unless specifically indicated to the contrary. Thus, if a class of components or residues A, B, and C are disclosed as well as a class of components or residues D, E, and F and an example of a combination compound A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Chromium Compounds

Disclosed herein, in one aspect, are compounds that comprise one or more chromium atoms and one or more fatty acids. According to the methods disclosed herein, such compounds can be administered to a subject and provide numerous health benefits, as described more fully below.

Many fatty acids are healthy oils that can serve as suitable vehicles for delivering various nutraceuticals such as vitamins, coenzymes, and phytosterols, and particularly for delivering small chemical entities such as, minerals, metals, and trace elements like chromium. This can be achieved either by a simple physical mixing, sometimes involving sophisticated technologies such as nanoparticling, or by a chemical bond. The use of oils and their concentrates with proven health benefits, such as those with a high content of omega-3 fatty acids, can add to the functionality of the product. The product can then becomes bi-functional by combining both the activity of the original substance to be delivered (e.g., chromium), with well known cardiovascular benefits of healthy oils (e.g., omega-3 fatty acids). (See Dyrberg, et al., In: ω-3 Fatty Acids: Prevention and Treatment of Vascular Disease. Kristensen, et al., eds., Bi & Gi Publ., Verona-Springer-Verlag, London, pp. 217-26, 1995; O'Keefe and Harris, Am J Cardiology 85:1239-41, 2000, which are incorporated by reference herein for their teachings of fatty acids and omega-3 fatty acids). Therefore, the disclosed compounds and compositions can be beneficial because they combine chromium with fatty acids (e.g., those derived from fish oils and those containing omega-3 concentrates).

In one aspect, the disclosed compounds comprise one or more chromium atoms bonded to one or more fatty acid residues. By “bonded,” or other forms of the word such as “bonds” or “bound,” is meant any type of interaction between atoms in which there is a donation, acceptance, or sharing of electrons, or an electrostatic interaction. With the disclosed compounds it can be difficult to determine with specificity the exact type of bonding that exists between a given chromium atom and a given fatty acid residue. Some examples of bonds that can exists in the compounds disclosed herein include, but are not limited to, covalent bonds, ionic bonds, dative bonds, multi-center bonds (e.g., bonds designated “μ” (mu) or “η” (eta)), an interaction between σ and/or π donors and acceptors, and an interaction between Lewis acids and bases (e.g., coordinate covalent bonds).

The term “residue” as used herein refers to the moiety that is the resulting product of the specified chemical species in a particular reaction scheme or subsequent formulation or chemical product, regardless of whether the moiety is actually obtained from the specified chemical species. For example, an “unsaturated fatty acid residue” refers to the moiety which results when an unsaturated fatty acid participates in a particular reaction (e.g., the residue can be an unsaturated fatty acyl group RCO— or acyloxyl group RCOO—). In this case, the unsaturated fatty acid residue is “derived” from the unsaturated fatty acid. It is understood that this moiety can be obtained by a reaction with a species other than the specified unsaturated fatty acid, for example, by a reaction with an unsaturated fatty acid chloride, ester, or anhydride.

### General Fatty Acids and Residues

The disclosed compounds can comprise one or more fatty acids or residues thereof. By “fatty acid” is meant a carboxylic acid with at least 10 carbon atoms. In one aspect, the fatty acids and residues thereof can comprise at least 10, at least 12, at least 14, at least 16, at least 18, or at least 20 carbon atoms. In some specific examples, the fatty acids and residues thereof can contain 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 carbon atoms, where any of the stated values can form an upper or lower endpoint when appropriate. In other examples, the fatty acids and residues thereof can comprise a mixture of fatty acids and residues thereof having a range of carbon atoms. For example, the fatty acids and residues thereof can comprise from about 10 to about 40, from about 12 to about 38, from about 14 to about 36, from about 16 to about 34, from about 18 to about 32, or from about 20 to 30 carbon atoms.

The fatty acids and residues thereof suitable for use herein can be saturated, unsaturated, or a mixture of saturated and unsaturated fatty acids. By “saturated” is meant that the molecule or residue contains no carbon-carbon double or triple bounds. By “unsaturated” is meant that the molecule or residue contains at least one carbon-carbon double or triple bond. In one specific example, the unsaturated fatty acid residue is not derived solely from oleic acid.

The fatty acids and residues thereof that can be used in the disclosed compounds and methods can be derived from any source. In one specific example, the fatty acids and residues thereof can be derived from fish oil. Such oils typically contain mixtures of saturated and unsaturated fatty acids, but can be processed to result in a particular mixture of fatty acids (e.g., containing all saturated, all unsaturated, mixtures of both, or mixtures with fatty acids of a certain chain length or range of chain lengths). Any fish oil can be used in the disclosed compounds and methods. Specific examples of suitable fish oils include, but are not limited to, Atlantic fish oils, Pacific fish oils, Mediterranean fish oils, light pressed fish oil, alkaline treated fish oil, beat treated fish oil, light and heavy brown fish oil, tuna oil, sea bass oil, halibut oil, spearfish oil, barracuda oil, cod oil, menhaden oil, sardine oil, anchovy oil, capelin oil, Atlantic cod oil, Atlantic herring oil, Atlantic mackerel oil, Atlantic menhaden oil, salmonids oil, shark oil, and the like.

Saturated Fatty Acids

Examples of specific saturated fatty acids and residues thereof that are suitable for the compounds and methods disclosed herein include, but are not limited to, capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

Unsaturated Fatty Acids

The unsaturated fatty acids and residues thereof that are suitable for the compounds and methods disclosed herein can comprise at least one unsaturated bond (i.e., a carbon-carbon double or triple bond). In one example, the unsaturated fatty acids and residues thereof can comprise at least 2, at least 3, at least 4, at least 5, at least 6, at least 7, at least 8, at least 9, or at least 10 carbon-carbon double bonds, triple bonds, or any combination thereof. In another example, the unsaturated fatty acids or residues thereof can comprise 1, 2, 3, 4, 5, 6, 7, 8, 9, or 10 unsaturated bonds, where any of the stated values can form an upper or lower endpoint when appropriate.

Monoene Acids and Residues

In one aspect, the unsaturated fatty acids or residues thereof can comprise one carbon-carbon double bond (i.e., a monoene acid or residue). Examples of unsaturated fatty acids and residues thereof that are suitable for the compounds and methods disclosed herein include, but are not limited to, those in the following Table 1.

TABLE 1
Examples of Monoenes

| Total number of carbon | Carbon number where double bond begins. |
|---|---|
| atoms in the fatty acid or | (“c” denotes a cis double bond; “t” denotes a |
| residue chain. | trans double bond) |
| 10 | 4c |
| 12 | 4c |
| 14 | 4c and 9c |
| 16 | 3t, 4c, 5t, 6c, 6t, 9c (palmitooleic), and 11c |
| 18 | 3t, 5c, 5t, 6c (petroselinic), 6t, 9c (oleic), 10c, |
| | 11c (cis-vaccenic), 11t (vaccenic), and 13c |
| 20 | 5c, 9c (gadolenic), 11c, 13c, and 15c |
| 22 | 5c, 11c (cetoleic), 13c (erucic), and 15c |
| 24 | 15c (selacholeic, nervonic) |
| 26 | 9c, and 17c (ximenic) |
| 28 | 9c, 19c (lumequic) |
| 30 | 21c |

Polyene Acids and Residues (Methylene Interrupted)

In another aspect, the unsaturated fatty acids and residues thereof can comprise at least two unsaturated bonds (e.g., polyene acids or residues). In some examples, the unsaturated fatty acids and residues thereof can comprise at least one pair of methylene interrupted unsaturated bonds. By “methylene interrupted unsaturated bond” is meant that one carbon-carbon double or triple bond is separated from another carbon-carbon double or triple bond by at least one methylene group (i.e., CH₂). Specific examples of unsaturated fatty acids that contain at least one pair of methylene interrupted unsaturated bonds include, but are not limited to, the n-1 family derived from 9, 12, 15-16:3; n-2 family derived from 9, 12, 15-17:3, 15:3, 17:3, 17:4, 20:4; n-3 family derived from 9, 12, 15-18:3, 15:2, 15:3, 15:4, 16:3, 16:4, 18:3 (α-linolenic), 18:4, 18:5, 20:2, 20:3, 20:4; 20:5 (EPA), 21:5, 22:3, 22:5 (DPA), 22:6 (DHA), 24:3, 24:4, 24:5, 24:6, 26:5, 26:6, 28:7, 30:5; n-4 family derived from 9, 12-16:2, 16:2, 16:3, 18:2, 18:3; n-5 family derived from 9, 12-17:2, 15:2, 17:2, 17:3, 19:2, 19:4, 20:3, 20:4 21:4, 21:5; n-6 family derived from 9, 12-18:2, 15:2, 16:2, 18:2 (linoleic acid), 18:3 (γ-linolenic acid); 20:2, 20:3, 20:4 (arachidonic acid), 22:2, 22:3, 22:4 (adrenic acid), 22:5, 24:2, 24:4, 25:2, 26:2, 30:4; n-7 family derived from 9-16:1, 15:2, 16:2, 17:2, 18:2, 19:2; n-8 family derived from 9-17:1, 15:2, 16:2, 17:2, 18:2, 19:2; n-9 family derived from 9-18:1, 17:2, 18:2, 20:2, 20:3, 22:3, 22:4; n-11 family 19:2, and the n-12 family 20:2.

In the above paragraph, the compounds are identified by referring first to the “n-x family,” where x is the position in the fatty acid where the first double bond begins. The numbering scheme begins at the terminal end of the fatty acid, where, for example, the terminal CH₃ group is designated position 1. In this sense, the n-3 family would be an omega-3 fatty acid, as described herein. The next number identifies the total number of carbon atoms in the fatty acid. The third number, which is after the colon, designates the total number of double bonds in the fatty acid. So, for example, in the n-1 family, 16:3, refers to a 16 carbon long fatty acid with 3 double bonds, each separated by a methylene, wherein the first double bond begins at position 1, i.e., the terminal end of the fatty acid. In another example, in the n-6 family, 18:3, refers to an 18 carbon long fatty acid with 3 methylene separated double bonds beginning at position 6, i.e., the sixth carbon from the terminal end of the fatty acid, and so forth.

Some other examples are fatty acids and residues thereof that contain at least one pair of unsaturated bonds interrupted by more than one methylene group. Suitable examples of these acids and residues thereof include, but are not limited to, those in the following Table 2:

TABLE 2
Examples of Polyene Acids and Residues with Double Bonds
Interrupted by Several Methylene Units

| Total number of carbon | Carbon number where double bond begins. |
|---|---|
| atoms in the fatty acid or | (“c” denotes a cis double bond; “t” denotes a |
| residue chain. | trans double bond) |
| 18 | 5, 9 |
| | 5, 11 |
| | 2t, 9, 12 |
| | 3t, 9, 12 |
| | 5t, 9, 12 |
| | 5, 9, 12 |
| | 5, 11, 14 |
| | 3t, 9, 12, 15 |
| | 5, 9, 12, 15 |
| 20 | 5, 11 |
| | 5, 13 |
| | 7, 11 |
| | 7, 13 |
| | 5, 11, 14 |
| | 7, 11, 14 |
| | 5, 11, 14, 17 |
| 22 | 5, 11 |
| | 5, 13 |
| | 7, 13 |
| | 7, 15 |
| | 7, 17 |
| | 9, 13 |
| | 9, 15 |

Polyene Acids and Residues (Conjugated)

Still other examples of unsaturated fatty acids and residues thereof that are suitable for use in the compounds and methods disclosed herein are those that contain at least one conjugated unsaturated bond. By “conjugated unsaturated bond” is meant that at least one pair of carbon-carbon double and/or triple bonds are bonded together, without a methylene (CH₂) group between them (e.g., —CH═CH—CH═CH—). Specific examples of unsaturated fatty acids that contain conjugated unsaturated bonds include, but are not limited to, those in the following Table 3.

TABLE 3
Examples of Conjugated Polyene Acids and Residues

| Total number of carbon | Carbon number where double bond begins. |
|---|---|
| atoms in the fatty acid or | (“c” denotes a cis double bond; “t” denotes a |
| residue chain. | trans double bond) |
| 10 | 2t, 4t, 6c |
| | 2c, 4t, 6t |
| | 3t, 5t, 7c |
| | 3c, 5t, 7t |
| 12 | 3, 5, 7, 9, 11 |
| 14 | 3, 5, 7, 9, 11 |
| 18 | 10t, 12t |
| | 8c, 10t, 12c (jacaric) |
| | 8t, 10t, 12c (calendic) |
| | 8t, 10t, 12t |
| | 9t, 11t, 13c (catalpic) |
| | 9c, 11t, 13t (α-eleostearic) |
| | 9c, 11t, 13c (punicic) |
| | 9t, 11t, 13t (β-eleostearic) |
| | 9c, 11t, 13t, 15c (α-parinaric) |
| | 9t, 11t, 13t, 15t (β-parinaric) |

Omega-3 Fatty Acids

Omega-3 fatty acids are certain unsaturated fatty acids that are particularly useful in the compounds and methods disclosed herein. Omega-3 fatty acids not only exhibit proven effects on lowering serum triglyceride levels, but they have strong connection to diabetes. For instance, docosahexaenoic acid (DHA) also has a strong insulin permeability enhancement effect, and it is viewed as a potential absorption enhancer for intestinal delivery of insulin (Onuki, et al., Int J Pharm 198:147-56, 2000). DH-A intake prevents certain biochemical processes that originate from insulin deficiency (Ovide-Bordeaux and Grynberg, Am J Physiol Regul Integr Comp Physiol 286:R519-27, 2003) and both DHA and EPA (eicosapentaenoic acid) significantly increase fasting insulin levels (Mori, et al., Am J Clin Nutr 71:1085-94, 2000).

An omega-3 fatty acid is an unsaturated fatty acid that contains as its terminus CH₃—CH₂—CH═CH—. Specific examples of omega-3 fatty acids that are suitable for use herein include, but are not limited to, linolenic acid (18:3ω3), octadecatetraenoic acid (18:4ω3), eicosapentaenoic acid (20:5ω3) (EPA), docosahexaenoic acid (22:6ω3) (DHA), docosapentaenoic acid (22:6ω3) (DPA), derivatives thereof and mixtures thereof.

In still other examples, the unsaturated fatty acids or residues thereof can be derived from a compound comprising the following formula:

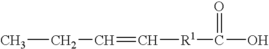

wherein R¹ is a C₃-C₄₀ alkyl or alkenyl group comprising at least one double bond. The term “alkane” or “alkyl” as used herein is a saturated hydrocarbon group. The term “alkene” or “alkenyl” as used herein is a hydrocarbon group of at least 2 carbon atoms with a structural formula containing at least one carbon-carbon double bond. Asymmetric structures such as (AB)C═C(CD) are intended to include both the E and Z isomers (cis and trans). This may be presumed in structural formulae herein wherein an asymmetric alkene is present, or it may be explicitly indicated by the bond symbol C═C. In a further example, R¹ can be a C₅-C₃₈, C₆-C₃₆, C₈-C₃₄, C₁₀-C₃₂, C₁₂-C₃₀, C₁₄-C₂₈, C₁₆-C₂₆, or C₁₈-C₂₄ alkenyl group. In yet another example, the alkenyl group of R¹ can have from 2 to 6, from 3 to 6, from 4 to 6, or from 5 to 6 double bonds. Still further, the alkenyl group of R¹ can have from 1, 2, 3, 4, 5, or 6 double bonds, where any of the stated values can form an upper or lower endpoint when appropriate.

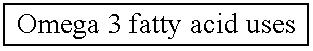

Omega-3 fatty acids are vital to everyday life and function. For example, the beneficial effects of omega-3 fatty acids like cis-5,8,11,14,17-eicosapentaenoic acid (EPA) and cis-4,7,10,13,16,19-docosahexaenoic acid (DHA) on lowering serum triglycerides are well established. These compounds are also known for other cardioprotective benefits such as preventing cardiac arrhythmias, stabilizing atherosclerotic plaques, reducing platelet aggregation, and reducing blood pressure. See e.g., Dyrberg et al., In: Omega-3 Fatty Acids Prevention and Treatment of Vascular Disease. Kristensen et al., eds., Bi & Gi Publ., Verona-Springer-Verlag, London, pp. 217-26, 1995; O'Keefe and Harris, Am. J. Cardiology 2000, 85:1239-41; Radack et al., “The effects of low doses of omega-3 fatty acid supplementation on blood pressure in hypertensive subjects: a randomized controlled trial.” Arch. Intern. Med. 1991, 151:1173-80; Harris, “Extending the cardiovascular benefits of omega-3 fatty acids.” Curr Atheroscler Rep 2005, 7:375-80; Holub, “Clinical nutrition: 4 omega-3 fatty acids in cardiovascular care.” CMAJ 2002, 166(5):608-15. Indeed, the American Heart Association has also reported that omega-3 fatty acids can reduce cardiovascular and heart disease risk. Other benefits of omega-3 fatty acids are those related to the prevention and/or treatment of inflammation and neurodegenerative diseases, and to improved cognitive development. See e.g., Sugano and Michihiro, “Balanced intake of polyunsaturated fatty acids for health benefits.” J. Oleo Sci. 2001, 50(5):305-11.

The fatty acids EPA and DHA can be synthesized in the human body from α-linolenic acid (18:3); however, the conversion rate from this precursor molecule is limited (Muskiet et al., “Is docosahexaenoic acid (DRA) essential? Lessons from DI-HA status regulation, our ancient diet, epidemiology and randomized controlled trials.” J Nutr 2004, 134(1):183-6). Accordingly, EPA and DHA in the body are primarily derived from dietary sources (e.g. oily fish). Diets rich in fish oils are known to have many beneficial effects for heart disease, cancer, arthritis, allergies, and other chronic diseases. Epidemiological clinical trials have shown that increasing the dietary intake of omega-3 fatty acids, in the form of fish or of fish oil supplements, may reduce various risk factors associated with cardiovascular disease. See e.g., The American Heart Association, Scientific Statement, “Fish Consumption, Fish Oil, Omega-3 Fatty Acids and Cardiovascular Disease,” November 2002; Appel et al., “Does supplementation of diet with ‘fish oil’ reduce blood pressure? A meta-analysis of controlled clinical trials.” Arch. Intern. Med. 1993, 153(12):1429-1438; GISSI-Prevenzione Investigators. “Dietary supplementation with omega-3 polyunsaturated fatty acids and vitamin E after myocardial infarction: results of the GISSI-Prevenzione trial.” Lancet 1999, 354:447-55.

Despite the strong evidence for the benefit of omega-3 fatty acids like EPA and DHA in prevention of cardiovascular disease, the average daily consumption of these fatty acids by North Americans is estimated to be between 0.1 to 0.2 grams, compared to a suggested daily intake of 0.65 grams to confer benefit (Webb, “Alternative sources of omega-3 fatty acids.” Natural Foods Merchandiser 2005, XXVI(8):40-4). Since altering dietary patterns of populations is difficult and many people do not like to eat fish, dietary supplementation with EPA and DHA is an important approach to addressing this problem. Unfortunately, many supplements of omega-3 fatty acids are sensitive to oxidation and can be foul smelling and tasting. Further, compliance with dietary supplement regimens requires discipline, which is often wanting.

Thus, disclosed are methods for lowering, reducing, or treating triglycerides in a subject comprising administering the disclosed compositions including the conjugated fatty acids, such as the omega 3 fatty acids to the subject, for example a subject in need of lowering, reducing, treating its triglyceride level or desirous of lowering, reducing, or treating its triglyceride level.

Also disclosed are methods for lowering, reducing, or treating depression in a subject comprising administering the disclosed compositions including the conjugated fatty acids, such as the omega 3 fatty acids to the subject, for example a subject in need of toweling, reducing, treating its depression or desirous of lowering, reducing, or treating its depression.

Also disclosed are methods for lowering, reducing, or treating inflammation in a subject comprising administering the disclosed compositions including the conjugated fatty acids, such as the omega 3 fatty acids to the subject, for example a subject in need of lowering, reducing, treating its inflammation or desirous of lowering, reducing, or treating its inflammation.

Also disclosed are methods for lowering, reducing, or treating blood pressure in a subject comprising administering the disclosed compositions including the conjugated fatty acids, such as the omega 3 fatty acids to the subject, for example a subject in need of lowering, reducing, treating its blood pressure or desirous of lowering, reducing, or treating its blood pressure.

Also disclosed are methods for lowering, reducing, or treating arrhythmias in a subject comprising administering the disclosed compositions including the conjugated fatty acids, such as the omega 3 fatty acids to the subject, for example a subject in need of lowering, reducing, treating its arrhythmias or desirous of lowering, reducing, or treating its arrhythmias.

Also disclosed are methods for promoting or increasing visual acuity or cognitive development in a subject comprising administering the disclosed compositions including the conjugated fatty acids, such as the omega 3 fatty acids to the subject, for example a subject in need of promoting or increasing its visual acuity or cognitive development or desirous of promoting or increasing its visual acuity or cognitive development or where it is desired that the subject increase its visual acuity or cognitive development. Also disclosed are methods, such as these for increasing infant development.

By desirous is met that the subject, for example, is aware of the need of the affect, such as by taking an assay or being diagnosed. By desired is met that possibly someone else, such as a physician or even a parent, for example, in the case of an infant is aware of the need for the affect because, of for example, a diagnosis, for the subject.

Exemplary Unsaturated Fatty Acids

Some specific examples of unsaturated fatty acids and residues derived therefrom that can be used in the compounds and methods disclosed herein include, but are not limited to linoleic acid, linolenic acid, γ-linolenic acid, arachidonic acid, mead acid, stearidonic acid, α-eleostearic acid, eleostearic acid, pinolenic acid, docosadienic acid, docosatetraenoic acid, docosapentaenoic acid, docosahexaenoic acid, octadecadienoic acid, octadecatrienoic acid, eicosatetraenoic acid, eicosapentaenoic, or any combination thereof. In one aspect, the unsaturated fatty acid residue can be derived from eicosapentacnoic acid 20:5ω3 (EPA), docosahexaenoic acid 22:6ω3 (DHA), docosapentaenoic acid 22:5ω3 (DPA), and any combination thereof.

Unsaturated Fatty Acids with Triple Bonds

Additional examples of suitable unsaturated fatty acids and residues thereof which are suitable in the disclosed compounds and methods include, but are not limited to, allenic and acetylenic acids, such as, C14: 2, 4, 5; C18: 5, 6 (laballenic); 5, 6, 16 (lamenallenic); C18: 6a (tarinic); 9a; 9a, 11t (ximenynic); 9a, 11a; 9a, 11a, 13c (bolekic); 9a, 11a, 13a, 15e, 8a, 10t (pyrulic) 9c, 12a (crepenynic); 9c, 12a, 14c (dehydrocrepenynic acid); 6a, 9c, 12c; 6a, 9c, 12c, 15c, 8a, 11c, 14c and corresponding Δ17e derivatives, 8-OH derivatives, and Δ17e, 8-OH derivatives.

Additional Fatty Acids

Branched-chain acids, particularly iso-acids and anteiso acids, polymethyl branched acids, phytol based acids (e.g., phytanic, pristanic), furanoid acids are also suitable fatty acids, including the residues derived therefrom, for use in the compounds and methods disclosed herein.

Still further, suitable fatty acids and residues thereof include, but are not limited to, cyclic acids, such as cyclopropane fatty acids, cyclopropene acids (e.g., lactobacillic), sterulic, malvalic, sterculynic, 2-hydroxysterculic, aleprolic, alepramic, aleprestic, aleprylic alepric, hydnocarpic, chaulmoogric hormelic, manaoic, gorlic, oncobic, cyclopentenyl acids, and cyclohexylalkanoic acids.

Hydroxy acids, particularly butolic, ricinoleic, isoricinoleic, densipolic, lesquerolic, and auriolic are also suitable fatty acids that can be used in the compounds and methods disclosed herein.

Epoxy acids, particularly epoxidated C18:1 and C18:2, and furanoid acids arc further examples of fatty acids that can be used in the disclosed compounds and methods.

Metal Atoms

The compounds disclosed herein comprise one or more chromium atoms. For example, the disclosed compounds can exists in various structures comprising from 1 to 6 chromium atoms, from 1 to 3 chromium atoms, from 1 to 2 chromium atoms, or from 2 to 3 chromium atoms. In another example, the disclosed compounds can have 3 chromium atoms. The chromium atoms in any of the disclosed compounds can be either Cr(II) or Cr(III). Further still, the disclosed compounds can have both Cr(II) and Cr(III).

The disclosed compounds can also comprise one or more non-chromium atoms bonded to either one or more of the chromium atoms, one or more of the fatty acid residues, or both. By “non-chromium atom” is meant a transition metal, alkaline metal, alkaline earth metal, rare earth metal, or metalloid. Examples of non-chromium atoms that can be present in the disclosed compounds include, but are not limited to, lithium, sodium, potassium, beryllium, magnesium, calcium, barium, scandium, titanium, vanadium, manganese, iron, cobalt, nickel, copper, zinc, yttrium, zirconium, niobium, molybdenum, technetium, ruthenium, rhodium, palladium, silver, cadmium, aluminum, gallium, indium, tin, antimony, tantalum, tungsten, lanthanum, and any combination thereof.

Hydrates

As is known in the art, water can become associated with a metal-containing compound such as the compounds disclosed herein. These interactions can involve one or several water molecules. For example, the disclosed compounds can further comprise one or more water molecules bonded to the chromium atom. The term “bonded” is used here as it is above and includes any sharing, donation, acceptance of electrons or electrostatic interaction. In some examples, the disclosed compounds can be bonded to 1, 2, 3, 4, 5, 6, 7, 8, 9, 10, 11, 12, 13, 14, 15, 16, 17, 18, 19, 20, 21, 22, 23, 24, 25, 26, 27, 28, or more water molecules, where any of the stated values can form an upper or lower endpoint when appropriate.

Exemplary Compounds

The structure of the metal-containing compounds will vary depending upon the selection of the chromium compound and fatty acid. Often, one or more distinct structures can be present in a given composition. Further, one specific form or complex can, under various conditions, convert into another. It should be understood that the disclosed compounds can comprise one or more of the disclosed formula or some species or variant thereof.

The various structures and complexes that can exist between chromium and a fatty acid, as disclosed herein, can be illustrated by considering chromium and acetic acid. For example, chromium(II) acetate, Cr₂(CH₃COO)₄.2H₂O can be prepared from chromous chloride and sodium acetate:

2CrCl₂+4CH₃COONa→Cr₂(CH₃COO)₄+4NaCl

Hexaaquachromium (III) acetate, [Cr(H₂O)₆](CH₃COO)₂, can be prepared from trihydrate of chromium (III) hydroxide:

Cr(OH)₃.3H₂O+3CH₃COOH→[Cr(H₂O)₆](CH₃COO)₃

Another Cr(III) acetate, [Cr(H₂O)₆]/(CH₃COO)₃H₂O, can be produced from hydrous chromic oxide or from [Cr(H₂O)₄(OH)₂]₂SO₄ and acetic acid (Kleinberg J, Unfamiliar Oxidation States and Their Stabilization. Lawrence, Kans., University of Kansas Press, 1950, which is incorporated by reference herein for its teaching of chromium compounds and method of preparing them).

Chromium acetate solutions are not simple. After standing an hour, the solutions contain monoaceto diacetic acid, [Cr(OH)₂(CH₃COO)](CH₃COOH)₂, which has not been isolated in a solid form (Abegg and Auerbach, Handbch der Anorganischen Chemie, Vol. 4 Leipzig, Verlag S Hirzel, 1921, which is incorporated by reference herein for its teaching of chromium compounds and method of preparing them). By concentrating the solution over concentrated sulfuric or acetic acid, violet plates of Cr(CH₃COO)₂.H₂O separate out. These crystals lose one molecule of acetic acid in the air and are only slightly water soluble. Their water solutions contain chromic diaceto acetic acid, [Cr(OH)CH₃—COO](CH₃COOH).

A green chromium diacetic acid [Cr₂O(CH₃COO)₄](CH₃COOH)₂ is also known (Diserens, The chemical technology of dyeing and printing. New York, Reinhold, 1948).

Acetic acid and an excess of hydrous chromic oxide leads to a violet crystalline water soluble powder, Cr(OH)(CH₃COO)₂ (Abegg and Auerbach, Handbch der Anorganischen Chemie, Vol. 4 Leipzig, Verlag S Hirzel, 1921).

Another complex acetate ion is [Cr₃(OH)(H₂O)/(CH₃COO)₆]₃⁺ and the best known acetate salts are:

[Cr₃(OH)(H₂O)/(CH₃COO)₆](CH₃COO)₂.3H₂O—green
[Cr₃(OH)(H₂O)/(CH₃COO)₆](CH₃COO)₂.1H₂O—green
[Cr₃(OH)(H₂O)/(CH₃COO)₆]2CrO₄(CH₃COO)₂.8H₂O—dark green
[Cr₃(OH)(H₂O)/(CH₃COO)₆]CrO₄.6H₂O—dark green
[Cr₃(H₂O)₂/(CH₃COO)₆](CH₃COO)₃.H₂O—green
[Cr₃(H₂O)₂/(CH₃COO)₆]Cr₂O₇(CH₃COO)—dark green

### [Cr₃(H₂O)₂/(CH₃COO)₆]CrO₄(CH₃COO).2.5H₂O

Under certain evaporation conditions [Cr₃(OH)(H₂O)/(CH₂COO)₆](CH₃COO)₂.1H₂O forms a violet solution from which Cr₆(CH₃COO)₁₂(OH)₆.24H₂O precipitate. In some cases the product has only 12H₂O. By treatment with acetic acid, Cr₆(CH₃COO)₁₆(OH)₂ with 6 or 12H₂O is formed and further treatment gives Cr₆(CH₃COO)₁₈.10H₂O. All of these “supercomplexes” are violet in color. In a solution, these complexes split in half:

Cr₆(CH₃COO)₁₂(OH)₆.24H₂O is converted to 2Cr₃(CH₃COO)₆(OH)₃.12H₂O
Cr₆(CH₃COO)₁₆(OH)₂.12H₂O becomes Cr₃(CH₃COO)₈(OH).6H₂O
Cr₆(CH₃COO)₁₈.10H₂O changes to Cr₃(CH₃COO)₉.5H₂O

Salts of [Cr₃(CH₃COO)₅(H₂O)(OH)₂]₂⁺ are also known, e.g.,

### [Cr₃(CH₃COO)₅(H₂O)(OH)₃](CH₃COO).11H₂O

### [Cr₃(CH₃COO)₅(H₂O)(OH)₃](CH₃COO).5H₂O

[Cr₃(CH₃COO)₅(H₂O)(OH)₃][Cr₃(CH₃COO)₅(H₂O)(OH)₂](CH₃COO)₃.10H₂O

### [Cr₃(CH₃COO)₅(H₂O)(OH)₂]CH₃COOH.H₂O

### [Cr₃(CH₃COO)₅(H₂O)(OH)₂]CH₃COOH.4H₂O

### [Cr₃(CH₃COO)₅(H₂O)(OH)₂](CH₃COOH)₂.2H₂O

Under certain conditions, the 6-aceto and 5-aceto-chromic complexes can partially loose their acetic acid resulting in [Cr₃(OH)₆(CH₃COO)₃] which reacts with acetic acid to give acetic acid richer complexes. (Abegg and Auerbach, Handbch der Anorganischen Chemie, Vol. 4 Leipzig, Verlag S Hirzel, 1921). In addition, the following compounds are known:

[Cr₃(CH₃COO)₃(OH)₅]Cr₃(CH₃COO)₃(OH)₄](CH₃COO)₃.28H₂O

### [Cr₃(CH₃COO)₃(OH)₃](CH₃COO)₃.7H₂O

[Cr₃(CH₃COO)₃(OH)₂][CH₃(CH₃COO)₃(OH)₃](CH₃COO)₇.10H₂O

### [Cr₃(CH₃COO)₃(OH)₂](CH₃COO)₄(CH₃COOH)₂

The above description shows the complexity observed with chromium and acetic acid (a two carbon acid). The complexity that results from compounds comprising chromium and fatty acids (e.g., an unsaturated fatty acid with at least 20 carbon atoms) is much greater. Thus, it is understood that there can be many various complexes or species that result when one or more chromium atoms are bonded to one or more fatty acids as disclosed herein. These variations are contemplated herein. Some specific examples of the disclosed compounds are provided in Table 4:

TABLE 4
Examples of Compounds Disclosed Herein

Cr₂(FA)₄•2H₂O
[Cr(H₂O)₆](FA)₂
[Cr(H₂O)₆]/(FA)₃•H₂O
[Cr(OH)₂(FA)](FA)₂
[Cr(OH)FA](FA)
[Cr₂O(FA)₄](FA)₂
Cr(OH)(FA)₂
[Cr₃(OH)(H₂O)/(FA)₆]₃⁺
[Cr₃(OH)(H₂O)/(FA)₆](FA)₂•3H₂O
[Cr₃(OH)(H₂O)/(FA)₆](FA)₂•1H₂O
[Cr₃(OH)(H₂O)/(FA)₆]2CrO₄(FA)₂•8H₂O
[Cr₃(OH)(H₂O)/(FA)₆]CrO₄•6H₂O
[Cr₃(H₂O)₂/(FA)₆](FA)₃•H₂O
[Cr₃(H₂O)₂/(FA)₆]Cr₂O₇(FA)
[Cr₃(H₂O)₂/(FA)₆]CrO₄(FA)•2.5H₂O
[Cr₃(OH)(H₂O)/(FA)₆](FA)₂•1H₂O
Cr₆(FA)₁₂(OH)₆•24H₂O
Cr₆(FA)₁₆(OH)₂•6H₂O
Cr₆(FA)₁₆(OH)₂•12H₂O
Cr₆(FA)₁₈•10H₂O
Cr₆(FA)₁₂(OH)₆•24H₂O
2Cr₃(FA)₆(OH)₃•12H₂O
Cr₆(FA)₁₆(OH)₂•12H₂O
Cr₃(FA)₈(OH)•6H₂O
Cr₃(FA)₉•5H₂O
[Cr₃(FA)₅(H₂O)(OH)₂]₂⁺
[Cr₃(FA)₅(H₂O)(OH)₃](FA)•11H₂O
[Cr₃(FA)₅(H₂O)(OH)₃](CH₃COO)•5H₂O
[Cr₃(FA)₅(H₂O)(OH)₃][Cr₃(FA)₅(H₂O)(OH)₂](FA)₃•10H₂O
[Cr₃(FA)₅(H₂O)(OH)₂]FA•H₂O
[Cr₃(FA)₅(H₂O)(OH)₂]FA•4H₂O
[Cr₃(FA)₅(H₂O)(OH)₂](FA)₂•2H₂O
[Cr₃(OH)₆(FA)₃]
[Cr₃(FA)₃(OH)₅]Cr₃(FA)₃(OH)₄](FA)₃•28H₂O
[Cr₃(FA)₃(OH)₃](FA)₃•7H₂O
[Cr₃(FA)₃(OH)₂][CH₃(FA)₃(OH)₃](FA)₇•10H₂O
[Cr₃(FA)₃(OH)₂](FA)₄(FA)₂
(FA = fatty acid (protonated or unprotonated))

Some additional examples of compounds disclosed and described herein can include, but are not limited to, compounds having the following Formula I:

[Cr₃(H₂O)₃(μ-fatty acid residue)₆(μ₃-O)]⁺ (I)

The symbol “μ” (mu) in the formula is commonly used in the art to refer to a bridging interaction between the ligand (e.g., unsaturated fatty acid) and the metal center(s) (e.g., chromium). Thus, in the above example, each fatty acid residue bridges two chromium atoms (μ) and the oxygen atom bridges three chromium atoms (μ₃).

In Formula I, the fatty acid residues can be any fatty acid as described herein. In one example, the fatty acid residue can be an unsaturated fatty acid residue as described herein. In another example, the fatty acid residues can be derived from fish oil. In another example, the fatty acid residues can comprise at least 20 carbon atoms in still another example, the fatty acid residue can be an unsaturated fatty acid residue comprising at least one pair of methylene interrupted unsaturated bonds. In yet another example, the fatty acid residues can be derived from linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof. In another example, the fatty acid residues can be derived from eicosapentaenoic acid 20:5ω3 (EPA), docosahexaenoic acid 22:6ω3 (DHA), docosapentaenoic acid 22:5ω3 (DPA), or any combination thereof.

In another aspect of the compounds characterized by Formula I, the fatty acid residue can be derived from an omega-3 fatty acid. For example, the compounds disclosed herein can be characterized by the following formula:

[Cr₃(H₂O)₃(μ-O₂C—R¹—CH═CH—CH₂—CH₃)₆(μ₃-O)]⁺

wherein R¹ is as described above, e.g., a C₃-C₄₀ alkenyl group comprising at least one double bond. R¹ can have, for example, from 2 to 6 double bonds or from 3 to 5 double bonds.

In a further aspect of the disclosed compounds, the compounds can comprise a fragment having the formula R²R³Cr—CrR⁴R⁵, wherein R²-R⁵ are the same or different fatty acid residues (e.g., unsaturated fatty acid residues). A “fragment,” as used herein refers to a portion or section of a compound or the entire compound. The disclosed fragments can have any variation of substituents; for example, the fragments can have substituents R²-R⁵ all being the same or all being different. In other examples, the disclosed fragments can have R² being the same as R³ (i.e., R²=R³), R² being the same as R⁴ (i.e., R²=R⁴), or R² being the same as R⁵ (i.e., R²=R⁵). In another example, R³═R⁴, R³=R⁵, R⁴=R⁵, R²=R³=R⁴, R²=R³=R⁵, R³=R⁴=R⁵, or R²=R³=R⁴=R⁵.

In one example, the disclosed fragments can have substituents R²-R⁵ comprising unsaturated fatty acid residues. The unsaturated fatty acid residues can be as described above. In another example, R²-R⁵ can be fatty acid residues derived from fish oil. In still another example, R²-R⁵ can be fatty acid residues comprising at least 20 carbon atoms. In yet another example, R²-R⁵ can be an unsaturated fatty acid residue comprising at least one pair of methylene interrupted unsaturated bonds.

In one aspect, R²-R⁵ can be fatty acid residues derived from an omega-3 fatty acid. For example, R²-R⁵ can be unsaturated fatty acid residues derived from a compound comprising the formula:

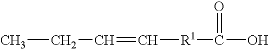

wherein R¹ is a C₃-C₄₀ alkyl or alkenyl group comprising at least one double bond. R¹ can be from, for example, 2 to 6 double bonds or from 3 to 5 double bonds. For example, R²-R⁵ can be fatty acid residues derived from linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof. In another example, R²-R⁵ can be fatty acid residues derived from cicosapentaenoic acid 20:5ω3 (EPA), docosahexaenoic acid 22:6ω3 (DHA), docosapentaenoic acid 22:5ω3 (DPA), or any combination thereof.

Additional Properties

The disclosed compounds can be, in one aspect, bioavailable. “Bioavailable” means that a compound is in a form that allows for it, or a portion of the amount administered, to be absorbed by, incorporated into, or otherwise physiologically available to a subject or patient to whom it is administered.

The biovailability of chromium compounds was shown in Catron (U.S. Pat. No. 5,846,581, which is incorporated by reference herein for its teachings of chromium compounds and their uses). Catron prepared chromium(III) compounds with short aliphatic chains containing from 3 to 7 carbon atoms. The dark green products are soluble in water and were found to be superior to chromium picolinate in effecting animal metabolism. This shows that chromium is bioavailable when in the form of organic acid salts. Methods to determine the bioavailability of drugs are well known to those of ordinary skill in the art.

In another aspect, the disclosed compounds can be liquids. For example, at room temperature, the disclosed compounds can be liquid.

### Methods of Making

Also disclosed herein are methods for preparing the disclosed compounds. In one aspect, the disclosed compounds can be prepared by reacting a chromium compound with one or more fatty acids (e.g., an unsaturated fatty acid) or the salt or ester thereof. In a particular example, the unsaturated fatty acid or the salt or ester thereof is not solely oleic acid. In another example, the reacting step does not involve a reducing agent.

Chromium Compounds

The chromium compounds that can be used in the disclosed methods can be obtained from any commercial source or can be synthesized by methods known in the art. Chromium compounds suitable for the methods described herein include chromium(III) and/or chromium(II) compounds. Such chromium compounds can be in the form of a hydrate.

Chromium(III) compounds suitable for use in the disclosed methods include, but are not limited to, CrCl₃, Cr(OH)₃, CrBr₃.6H₂O, CrF₃, CrF₃.4H₂O, CrCl₃, KCr(SO₄)₂.12H₂O, Cr₂(SO₄)₃.xH₂O, Cr₂S₃, [Cr(H₂O)₄Cl₂]Cl.2H₂O, Cr(H₂O)₆Cl₃, Cr₂O₃, Cr₂O₃.xH₂O, and CrPO₄.4H₂O, where x is an integer from 1 to 28, including mixtures and hydrates thereof.

Chromium(II) compounds suitable for use in the disclosed methods include, but are not limited to, CrCl₂, Cr(SO₄)₂, CrCl₂.4H₂O, CrS, and CrO, including hydrates thereof.

Fatty Acids

The chromium compound can be reacted with the fatty acid (e.g., unsaturated fatty acid). Any of the fatty acids disclosed and described herein can be used. In another aspect, the fatty acids can be in the acid chloride, ester, or anhydride form. Such derivatives of the fatty acids described above are considered as being disclosed herein.

In one example, the fatty acid can be derived from fish oil. In another example, the fatty acid can comprise at least 20 carbon atoms. In yet another example, the unsaturated fatty acid can comprise at least one pair of methylene interrupted unsaturated bonds. In a further example, the fatty acid can be an omega-3 fatty acid. In still another example, the unsaturated fatty acid can comprise the formula:

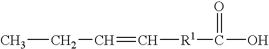

wherein R¹ is a C₃-C₄₀ alkyl or alkenyl group comprising at least one double bond. The substituent R¹ has from 2 to 6 double bonds

In some specific examples, the fatty acid can be linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof. In other specific examples, the fatty acid can comprise eicosapentaenoic acid 20:5ω3 (FPA), docosahexaenoic acid 22:6ω3 (DHA), docosapentaenoic acid 22:5ω3 (DPA), or any combination thereof.

Concentration

The reaction between the chromium compound and one or more fatty acids can take place under various conditions. For example, the reaction can take place neat. In another aspect, the reaction can take place in or more solvents. For example, the reaction can take place in an aqueous solvent, such as, but not limited to, water, aqueous hexane, aqueous ethanol, aqueous methanol, aqueous propanol, and the like. Other examples include diphasic systems containing an aqueous phase and an organic phase. In these systems, suitable organic phases can contain, for example, butanol, pentane, cyclopentane, hexane, cyclohexane, heptane, benzene, toluene, carbon tetrachloride, chloroform, methylene chloride, dichloroethane, ethyl acetate, ether, MEK, octane, diisopropyl ether, tri and tetrachlorethane, and the like. The amount of solvent used and the concentration of the chromium compound and/or fatty acid will depend on the particular compound being prepared, the type of chromium compound, the type of fatty acid, preference, and the like.

Temperature

The chromium compound can be reacted with the fatty acid or derivative thereof at any temperature sufficient to form a bond between the chromium atom of the chromium compound and the fatty acid. Typically, the reaction can take place at an elevated temperature. The precise elevated temperature can depend on the particular fatty acid being used, the particular chromium compound being used, the solvent, the amount or concentration of the reagents, preference, and the like. Suitable temperatures at which the chromium compound can be reacted with the fatty acid include, but are not limited to, from about 20 to about 200° C., from about 50 to about 220° C., from about 70 to about 240° C., from about 90 to about 260° C., or from about 110 to about 280° C. In other examples, the temperature of the reaction can be at about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 300° C., where any of the stated values can form an upper or lower endpoint when appropriate.

Non-Chromium Compounds

The disclosed method can further comprise reacting the fatty acid with a non-chromium compound. A non-chromium compound is one that contains a non-chromium atom, as described above. For example, a non-chromium atom can be a transition metal, alkaline metal, alkaline earth metal, rare earth metal, or metalloid. Examples of non-chromium atoms that can be present in a non-chromium compound reacted according to the disclosed methods include, but are not limited to, lithium, sodium, potassium, beryllium, magnesium, calcium, barium, scandium, titanium, vanadium, manganese, iron, cobalt, nickel, copper, zinc, yttrium, zirconium, niobium, molybdenum, technetium, ruthenium, rhodium, palladium, silver, cadmium, aluminum, gallium, indium, tin, antimony, tantalum, tungsten, lanthanum, and any combination thereof.

Also disclosed are products prepared by the disclosed methods.

Other Methods

The disclosed compounds can also be made by any other method known in the art. For example, the methods disclosed above for preparing various chromium acetates can be employed herein to prepare the chromium-fatty acid compounds disclosed herein.

In another suitable method, Czech researchers prepared chromium(III)-long chain fatty acids using a direct reaction involving hexaaquachromium trichloride. These salts were used as catalysts to produce monoacylglycerols (Janis, et al., Eur J Lipid Sci Technol 351-354, 2000, which is incorporated herein for its teaching of methods of preparing chromium compounds). The fatty acids, oleic (C18:1), palmitic (16:0) and stearic (18:0) acids were investigated for that purpose.

Other methods for preparing chromium compounds, which are suitable for preparing the compounds disclosed herein, are disclosed in U.S. Pat. No. 6,809,115 to Katz, et al.; U.S. Pat. No. 5,846,581 to Catron; and Hein and Herzog, Chromium, Molybdenum, Tungsten, Uranium. In: Handbook of Preparative Inorganic Chemistry. Vol. 2. Brauer G., Academic Press, New York, pp. 1334-1401, 1965, which are incorporated by reference herein for their teaching of chromium compounds and methods for their preparation.

### Supplements

Also disclosed herein are nutritional supplements. A nutritional supplement is any compound or composition that can be administered to or taken by a subject to provide, supply, or increase a nutrient(s) (e.g., vitamin, mineral, essential trace element, amino acid, peptide, nucleic acid, oligonucleotide, lipid, cholesterol, steroid, carbohydrate, and the like). In one aspect, disclosed herein are nutritional supplements comprising any of the compounds disclosed herein. For example, a nutritional supplement can comprise a chromium compound comprising one or more chromium atoms bonded to one or more fatty acid residues (e.g., [Cr₃(H₂O)₃(μ-fatty acid residue)₆(μ₃-O)]⁺). The fatty acid residues can be any fatty acid as disclosed herein (e.g., unsaturated or saturated fatty acid residues).

The nutritional supplement can comprise any amount of the compounds disclosed herein, but will typically contain an amount determined to supply a subject with a desired dose of chromium. The exact amount of compound required in the nutritional supplement will vary from subject to subject, depending on the species, age, weight and general condition of the subject, the severity of the dietary deficiency being treated, the particular mode of administration, and the like. Thus, it is not possible to specify an exact amount for every nutritional supplement. However, an appropriate amount can be determined by one of ordinary skill in the art using only routine experimentation given the teachings herein. In one specific example, a nutritional supplement can comprise from about 10 to about 3000 micrograms of chromium, from about 20 to about 1500 micrograms, from about 50 to about 200 micrograms. In another example, the nutritional supplement can comprise from about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 500, 600, 700, 800, 900, 1000, 1500, 2000, 2500, or 3000 micrograms of chromium, where any of the stated values can form an upper or lower endpoint when appropriate.

The nutritional supplement can also comprise other nutrient(s) such as vitamins other trace elements, minerals, and the like. Further, the nutritional supplement can comprise other components such as preservatives, antimicrobials, anti-oxidants, chelating agents, thickeners, flavorings, diluents, emulsifiers, dispersing aids, or binders.

The nutritional supplements are generally taken orally and can be in any form suitable for oral administration. For example, a nutritional supplement can typically be in a tablet, gel-cap, capsule, liquid, sachets, or syrup form.

### Pharmaceutical Formulation

Also, disclosed herein are pharmaceutical formulations. In one aspect, a pharmaceutical formulation can comprise any of the compounds disclosed herein with a pharmaceutically acceptable carrier. For example, a pharmaceutical formulation can comprise a chromium compound comprising one or more chromium atoms bonded to one or more fatty acids and a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) Gennaro, ed., Mack Publishing Company, Easton, Pa., 1995, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, antiinflammatory agents, anesthetics, and the like.

The pharmaceutical formulation can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids or binders may be desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Delivery Devices

Any of the compounds described herein can be incorporated into a delivery device. Examples of delivery devices include, but are not limited to, microcapsules, microspheres, nanospheres or nanoparticles, liposomes, noisome, nanoerythrosome, solid-liquid nanoparticles, gels, gel capsules, tablets, lotions, creams, sprays, emulsions, or powders. Other examples of delivery devices that are suitable for non-oral administration include pulmospheres. Examples of particular delivery devices useful herein are described below.

The disclosed compounds can be incorporated into liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The disclosed compositions in liposome form can contain, in addition to a compound disclosed herein, stabilizers, preservatives, excipients, and the like. Examples of suitable lipids are the phospholipids and the phosphatidyl cholines (lecithins), both natural and synthetic. Methods of forming liposomes are known in the art. See, e.g., Prescott, Ed., Methods in Cell Biology, Volume XIV, Academic Press, New York, p. 33 et seq., 1976, which is hereby incorporated by reference herein for its teachings of liposomes and their preparation.

In other examples, the liposomes can be cationic liposomes (e.g., DOTMA, DOPE, DC cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham, et al., Am J Resp Cell Mol Biol 1:95-100, 1989; Felgner, et al., Proc Natl Acad Sci USA 84:7413-7, 1987; and U.S. Pat. No. 4,897,355, which are incorporated by reference herein for their teachings of liposomes. As one example, delivery can be via a liposome using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. Liposomes where the diffusion of the compound or delivery of the compound from the liposome is designed for a specific rate or dosage can also be used.

As described herein, niosomes are delivery devices that can be used to deliver the compositions disclosed herein. Noisomes are multilamellar or unilamellar vesicles involving non-ionic surfactants. An aqueous solution of solute is enclosed by a bilayer resulting from the organization of surfactant macromolecules. Similar to liposomes, noisomes are used in targeted delivery of, for example, anticancer drugs, including methotrexate, doxorubicin, and immunoadjuvants. They are generally understood to be different from transferosomes, vesicles prepared from amphiphilic carbohydrate and amino group containing polymers, e.g., chitosan.

As described herein, nanoerythrosomes are delivery devices that can be used to deliver the compositions disclosed herein. Nanoerythrosomes are nano-vesicles made of red blood cells via dialysis through filters of defined pore size. These vesicles can be loaded with a diverse array of biologically active molecules, including proteins and the compositions disclosed herein. They generally serve as ideal carriers for antineoplastic agents like bleomycin, actinomycin D, but can be used for steroids, other lipids, etc.

Artificial red blood cells, as described herein, are further delivery devices that can be used to deliver the compositions disclosed herein. Artificial red blood cells can be generated by interfacial polymerization and complex emulsion methods. Generally, the “cell” wall is made of polyphtaloyl L-lysine polymer/polystyrene and the core is made of a hemoglobin solution from sheep hemolysate. Hemoglobin loaded microspheres typically have particle sizes of from about 1 to about 10 mm. Their size, flexibility, and oxygen carrying capacity is similar to red blood cells.

Solid-lipid nanoparticles, as described herein, are other delivery devices that can be used to deliver the compositions disclosed herein. Solid-lipid nanoparticles are nanoparticles, which are dispersed in an aqueous surfactant solution. They are comprised of a solid hydrophobic core having a monolayer of a phospholipid coating and are usually prepared by high-pressure homogenization techniques. Immunomodulating complexes (ISCOMS) are examples of solid-lipid nanoparticles. They are cage-like 40 nm supramolecular assemblies comprising of phospholipid, cholesterol, and hydrophobic antigens and are used mostly as immunoadjuvants. For instance, ISCOMs are used to prolong blood-plasma levels of subcutaneously injected cyclosporine.

Microspheres and micro-capsules, as described herein, are yet other delivery devices that can be used to deliver the compositions disclosed herein. In contrast to liposomal delivery systems, microspheres and micro-capsules typically do not have an aqueous core but a solid polymer matrix or membrane. These delivery devices are obtained by controlled precipitation of polymers, chemical cross-linking of soluble polymers, and interfacial polymerization of two monomers or high-pressure homogenization techniques. The encapsulated compound is gradually released from the depot by erosion or diffusion from the particles. Successful formulations of short acting peptides, such as LHRH agonists like leuprorelin and triptoreline, have been developed. Poly(lactide co-glycolide (PLGA) microspheres are currently used as monthly and three monthly dosage forms in the treatment of advanced prostrate cancer, endometriosis, and other hormone responsive conditions. Leuprolide, an LHRH superagonist, was incorporated into a variety of PLGA matrices using a solvent extraction/evaporation method. As noted, all of these delivery devices can be used in the methods disclosed herein.

Pulmospheres are still other examples of delivery devices that can be used herein. Pulmospheres are hollow porous particles with a low density (less than about 0.1 gm/mL). Pulmospheres typically have excellent re-dispersibility and are usually prepared by supercritical fluid condensation technology. Co-spray-drying with certain matrices, such as carbohydrates, human serum albumin, etc., can improve the stability of proteins and peptides (e.g., insulin) and other biomolecules for pulmonary delivery. This type of delivery could be also accomplished with micro-emulsions and lipid emulsions, which are ultra fine, thin, transparent oil-in-water (o/w) emulsions formed spontaneously with no significant input of mechanical energy. In this technique, an emulsion can be prepared at a temperature, which must be higher than the phase inversion temperature of the system. At elevated temperature the emulsion is of water-in-oil (w/o) type and as it cools at the phase inversion temperature, this emulsion is inverted to become o/w. Due to their very small inner phase, they are extremely stable and used for sustained release of steroids and vaccines. Lipid emulsions comprise a neutral lipid core (i.e., triglycerides) stabilized by a monolayer of amphiphilic lipid (i.e., phospholipid) using surfactants like egg lecithin triglycerides and miglyol. They are suitable for passive and active targeting.

There are other oral delivery systems under investigation that are based on osmotic pressure modulation, pH modulation, swelling modulation, altered density and floating systems, mucoadhesiveness etc. These formulations and time-delayed formulations to deliver drugs in accordance with circadian rhythm of disease that are currently in use or investigation can be applied for delivery of the compositions disclosed herein.

Microcapsules

In one aspect disclosed herein, the disclosed compounds can be incorporated into microcapsules. In one aspect, the microcapsule comprises an agglomeration of primary microcapsules and the chromium compounds described herein, each individual primary microcapsule having a primary shell, wherein the chromium compound is encapsulated by the primary shell, wherein the agglomeration is encapsulated by an outer shell. These microcapsules are referred to herein as “multicore microcapsules.”

In another aspect, described herein are microcapsules comprising a chromium compound, a primary shell, and a secondary shell, wherein the primary shell encapsulates the chromium compound, and the secondary shell encapsulates the loading substance and primary shell. These microcapsules are referred to herein as “single-core microcapsules.”

Optionally, other loading substances can be encapsulated with the chromium compound. The loading substance can be any substance that is not entirely soluble in the aqueous mixture. In one aspect, the loading substance is a solid, a hydrophobic liquid, or a mixture of a solid and a hydrophobic liquid. In another aspect, the loading substance comprises a grease, an oil, a lipid, a drug (e.g., small molecule), a biologically active substance, a nutritional supplement (e.g., vitamins), a flavour compound, or a mixture thereof. Examples of oils include, but are not limited to, animal oils (e.g., fish oil, marine mammal oil, etc.), vegetable oils (e.g., canola or rapeseed), mineral oils, derivatives thereof or mixtures thereof. The loading substance can be a purified or partially purified oily substance such as a fatty acid, a triglyceride or ester thereof, or a mixture thereof. In another aspect, the loading substance can be a carotenoid (e.g., lycopene), a satiety agent, a flavor compound, a drug (e.g., a water insoluble drug), a particulate, an agricultural chemical (e.g., herbicides, insecticides, fertilizers), or an aquaculture ingredient (e.g., feed, pigment).

In one aspect, the loading substance can be an omega-3 fatty acid. Examples of omega-3 fatty acids include, but are not limited to, α-linolenic acid (18:3ω3), octadecatetraenoic acid (18:4ω3), eicosapentaenoic acid (20:5ω3) (EPA), docosahexaenoic acid (22:6×3) (DHA), docosapentaenoic acid (22:5ω3) (DPA), eicosatetraenoic acid (20:4ω3), uncosapentaenoic acid (21:5ω3), docosapentacnoic acid (22:5ω3) and derivatives thereof and mixtures thereof. Many types of derivatives of omega-3 fatty acids are well known in the art. Examples of suitable derivatives include, but are not limited to, esters, such as phytosterol esters, branched or unbranched C₁-C₃₀ alkyl esters, branched or unbranched C₂-C₃₀ alkenyl esters, or branched or unbranched C₃-C₃₀ cycloalkyl esters such as phytosterol esters and C₁-C₆ alkyl esters. Sources of oils can be derived from aquatic organisms (e.g., anchovies, capelin, Atlantic cod, Atlantic herring, Atlantic mackerel, Atlantic menhaden, salmonids, sardines, shark, tuna, etc) and plants (e.g., flax, vegetables, etc) and microorganisms (e.g., fungi and algae).

In one aspect, the loading substance can contain an antioxidant. Examples of antioxidants include, but are not limited to, vitamin E, CoQ₁₀, tocopherols, lipid soluble derivatives of more polar antioxidants such as ascorbyl fatty acid esters (e.g., ascorbyl palmitate), plant extracts (e.g., rosemary, sage and oregano oils), algal extracts, and synthetic antioxidants (e.g., BHT, TBHQ, ethoxyquin, alkyl gallates, hydroquinones, tocotrienols).

A number of different polymers can be used to produce the shell layers of the single and multicore microcapsules. Examples of such polymers include, but are not limited to, a protein, a polyphosphate, a polysaccharide, or a mixture thereof. In another aspect, the shell material used to prepare the single- and multicore microcapsules further comprises In another aspect, the shell material used to prepare the single- and multicore microcapsules further comprises gelatin type A, gelatin type B, polyphosphate, gum arabic, alginate, chitosan, carrageenan, pectin, starch, modified starch, alfa-lactalbumin, beta-lactoglobumin, ovalbumin, polysorbiton, maltodextrins, cyclodextrins, cellulose, methyl cellulose, ethyl cellulose, hydropropylmethylcellulose, carboxymethylcellulose, milk protein, whey protein, soy protein, canola protein, albumin, chitin, polylactides, poly-lactide-co-glycolides, derivatized chitin, chitosan, poly-lysine, various inorganic-organic composites, or any mixture thereof. It is also contemplated that derivatives of these polymers can be used as well. In another aspect, the polymer can be kosher gelatin, non-kosher gelatin, Halal gelatin, or non-Halal gelatin.

In one aspect, one or more of the shell layers in the single and multicore microcapsules comprises gelatin having a Bloom number less than 50. This gelatin is referred to herein as “low Bloom gelatin.” The Bloom number describes the gel strength formed at 10° C. with a 6.67% solution gelled for 18 hours. In one aspect, the low Bloom gelatin has a Bloom number less than 40, less than 30, less than 20, or less than 10. In another aspect, the gelatin has a Bloom number of 45, 40, 35, 30, 25, 20, 15, 10, 9, 8, 7, 6, 5, 4, 3, 2, 1, or 0, where any two values can be used to produce a range. In another aspect, the low Bloom gelatin is in both the primary shell and the outer shell of the multicore microcapsule. In one aspect, the low Bloom gelatin is gelatin type A. In another aspect, the low Bloom gelatin is gelatin type A produced by Kenney & Ross Ltd., R.R. #3 Shelburne, NS Canada. In another aspect, gelatin having a Bloom number of zero is in both the primary shell and the outer shell of the multicore microcapsule.

In one aspect, the material used to make the shells of the single- or multicore microcapsules is a two-component system made from a mixture of two different types of polymers. In one aspect, the material is a complex coacervate between the polymer components. Complex coacervation is caused by the interaction between two oppositely charged polymers. In one aspect, the shell material used to produce the single and multicore microcapsules is composed of (1) low Bloom gelatin and (2) gelatin type B, polyphosphate, gum arabic, alginate, chitosan, carrageenan, pectin, carboxymethylcellulose, whey protein, soy protein, canola protein, albumin, or a mixture thereof. The molar ratio of the different polymers can vary. For example, the molar ratio of low Bloom gelatin to the other polymer component is from 1:5 to 15:1. For example, when low Bloom gelatin and polyphosphate are used, the molar ratio of low Bloom gelatin to polyphosphate is about 8:1 to about 12:1; when low Bloom gelatin and gelatin type B are used, the molar ratio is 2:1 to 1:2; and when low Bloom gelatin and alginate are used, the molar ratio is 3:1 to 8:1.

Processing aids can be included in the shell material (e.g., primary or outer shells). Processing aids can be used for a variety of reasons. For example, they may be used to promote agglomeration of the primary microcapsules, stabilize the emulsion system, improve the properties of the outer shells, control microcapsule size, and/or to act as an antioxidant. In one aspect, the processing aid can be an emulsifier, a fatty acid, a lipid, a wax, a microbial cell (e.g., yeast cell lines), a clay, or an inorganic compound (e.g., calcium carbonate). Not wishing to be bound by theory, these processing aids can improve the barrier properties of the microcapsules. In one aspect, one or more antioxidants can be added to the shell material. Antioxidant properties are useful both during the process (e.g. during coacervation and/or spray drying) and in the microcapsules after they are formed (i.e. to extend shelf-life, etc). Preferably a small number of processing aids that perform a large number of functions can be used. In one aspect, the antioxidant can be a phenolic compound, a plant extract, or a sulphur-containing amino acid. In one aspect, ascorbic acid (or a salt thereof such as sodium or potassium ascorbate) can be used to promote agglomeration of the primary microcapsules, to control microcapsule size and to act as an antioxidant. The antioxidant can be used in an amount of about 100 ppm to about 12,000 ppm, or from about 1,000 ppm to about 5,000 ppm. Other processing aids such as, for example, metal chelators, can be used as well. For example, ethylene diamine tetraacetic acid can be used to bind metal ions, which can reduce the catalytic oxidation of the loading substance.

In one aspect, the primary microcapsules (primary shells) have an average diameter of about 40 nm to about 10 μm, 0.1 μm to about 10 μm, 1 μm to about 10 μm, 1 μm to about 8 μm, 1 μm to about 6 μm, 1 μm to about 4 μm, or 1 μm to about 2 μm, or 1 μm. In another aspect, the multicore microcapsules can have an average diameter of from about 1 μm to about 2000 μm, 20 μm to about 1000 μm, from about 20 μm to about 100 μm, or from about 30 μm to about 80 μm. In another aspect, the single-core microcapsules have an outer diameter of from 1 μm to 2,000 μm.

The microcapsules described herein generally have a combination of high payload and structural strength. For example, payloads of loading substance can be from 20% to 90%, 50% to 70% by weight, or 60% by weight of the single or multicore microcapsules.

In one aspect, the methods disclosed in U.S. Patent Application Publication No. 2003/0193102, which is incorporated by reference in its entirety, can be used to encapsulate the chromium compounds described herein. It is also contemplated that one or more additional shell layers can be placed on the outer shell of the single or multicore microcapsules. In one aspect, the techniques described in International Publication No. WO 2004/041251 A1, which is incorporated by reference in its entirety, can be used to add additional shell layers to the single and multicore microcapsules.

### Targeted Delivery

The compounds disclosed herein can be targeted to a particular cell type, such as islets cells, via antibodies, receptors, or receptor ligands. The following references are examples of the use of this technology to target specific tissue (Senter, et al., Bioconjugate Chem 2:447-51, 1991; Bagsbawe, Br J Cancer 60:275-81, 1989; Bagshawe, et al., Br J Cancer 58:700-3, 1988; Senter, et al., Bioconjugate Chem 4:3-9, 1993; Battelli, et al., Cancer Immunol Immunother 35:421-5, 1992; Pietersz and McKenzie, Immunolog Reviews 129:57-80, 1992; and Roffler, et al., Biochem Pharmacol 42:2062-5, 1991): These techniques can be used for a variety of other specific cell types.

### Foodstuffs

Also disclosed herein are foodstuffs comprising any of the microcapsules and emulsions disclosed herein. By “foodstuff” is meant any article that can be consumed (e.g., eaten, drank, or ingested) by a subject. In one aspect, the microcapsules can be used as nutritional supplements to a foodstuff. For example, the microcapsules and emulsions can be loaded with vitamins, omega-3 fatty acids, and other compounds that provide health benefits. In one aspect, the foodstuff is a baked good, a pasta, a meat product, a frozen dairy product, a milk product, a cheese product, an egg product, a condiment, a soup mix, a snack food, a nut product, a plant protein product, a hard candy, a soft candy, a poultry product, a processed fruit juice, a granulated sugar (e.g., white or brown), a sauce, a gravy, a syrup, a nutritional bar, a beverage, a dry beverage powder, a jam or jelly, a fish product, or pet companion food. In another aspect, the foodstuff is bread, tortillas, cereal, sausage, chicken, ice cream, yoghurt, milk, salad dressing, rice bran, fruit juice, a dry beverage powder, rolls, cookies, crackers, fruit pies, or cakes.

### Methods of Use:

Chromium is an essential trace element involved in glucose, lipid, and protein metabolism. Deficiencies of chromium are often deleterious. Chromium deficiency has been linked to an increased risk of heart disease, diabetes, hypoglycemia, obesity, impaired metabolism and diminished longevity. Cardiovascular disease and diabetes alone account for about sixty percent of premature deaths in the USA annually, and death usually strikes these victims 10 to 20 years before they reach the average life span. The National Academy of Sciences has recommended an intake for humans of about 50 to 200 micrograms of trivalent chromium daily. It has been reported that 90% of adults fail to ingest even the minimum recommended amount.

Methods that can provide or supply chromium to a subject are therefore beneficial. As an example, Katz et al. discloses chromium compositions for treating insulin-dependent diabetes, improving insulin sensitivity, and reducing hyperlipidemia, including hypercholesterolemia (U.S. Pat. No. 6,809,115). Further, U.S. Pat. Nos. 5,087,623, 5,087,624, and 5,175,156, disclose the use of chromium picolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum HDL-cholesterol levels. Still further, Catron discloses chromium(III) compounds with short chain acids containing from 3 to 7 carbon atoms, which were found to be superior to chromium picolinate in effecting animal metabolism (U.S. Pat. No. 5,846,581). These references are incorporated by reference herein for their teachings of uses for chromium compounds.

The compounds disclosed herein also have a wide variety of uses. In the disclosed compounds, the one or more fatty acids are bonded to the one or more chromium atoms and are therefore an integral part of the complex. Thus, while not wishing to be bound by theory, it is believed that the fatty acids (e.g., DHA, DPA, and/or EPA) play at least two roles, i.e., they make Cr(III) biologically available and they also contribute with their inherent biological activity. Thus, the disclosed compounds (including the nutritional supplements, pharmaceutical formulations, delivery devices, and foodstuffs) can deliver (1) fatty acids (e.g., omega-3 fatty acids), lowering triglycerides and influencing diabetes related biochemistry, and (2) chromium, a trace element with a beneficial effect on serum cholesterol levels and diabetes.

In one particular aspect, disclosed herein are methods of supplementing essential trace elements in a subject by administering an effective amount of a chromium compound comprising one or more chromium atoms bonded to one or more fatty acid residues. In another aspect, disclosed herein are methods of lowering cholesterol levels, triglyceride levels, or a combination thereof in a subject by administering an effective amount of a chromium compound comprising one or more chromium atoms bonded to one or more fatty acid residues. In still another aspect, disclosed herein are methods of improving insulin sensitivity in a subject by administering an effective amount of a chromium compound comprising one or more chromium atoms bonded to one or more fatty acid residues. In a further aspect, disclosed herein are methods of reducing hyperglycemia in a subject by administering an effective amount of a chromium compound comprising one or more chromium atoms bonded to one or more fatty acid residues. In yet another aspect, disclosed herein are methods of reducing hypercholesterolemia in a subject by administering an effective amount of a chromium compound comprising one or more chromium atoms bonded to one or more fatty acid residues.

Also disclosed herein, in one aspect, are methods of reducing body fat in a subject by administering an effective amount of a chromium compound comprising one or more chromium atoms bonded to one or more fatty acid residues. In another aspect, disclosed herein are methods of promoting weight loss in a subject by administering an effective amount of a chromium compound comprising one or more chromium atoms bonded to one or more fatty acid residues. In still another aspect, disclosed herein are methods of treating or preventing diabetes in a subject by administering an effective amount of a chromium compound comprising one or more chromium atoms bonded to one or more fatty acid residues.

Also disclosed are methods, wherein the treating diabetes comprises reducing the blood glucose level in the subject. Also disclosed are methods, wherein the blood glucose level is reduced by at least 1, 2, 3, 4, 5, 6, 7, 8, 9, m10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 300, 400, 500, or 1000%.

Also disclosed are methods, wherein the blood glucose level is reduced by at least 1, 2, 3, 4, 5, 6, 7, 8, 9, 10, 11, 12, 13, 14, 15, 16, 17, 18, 19, 20, 21, 22, 23, 24, or 25 mM.

In the disclosed methods, the chromium compounds can be any of the chromium compounds disclosed herein. Also, the chromium compounds can be used neat or in combination with some other component. For example, the chromium compounds can be used in the disclosed methods in the form of any of the nutritional supplements disclosed herein. In another example, the chromium compounds can be used in the disclosed methods in the form of any of the pharmaceutical formulations disclosed herein. In still another example, the chromium compounds can be incorporated in any of the delivery devices disclosed herein, or incorporated into any foodstuff disclosed herein and used in the disclosed methods.

It is contemplated that the methods disclosed herein can be accomplished by administering various forms of the chromium compounds disclosed herein. For example, one can administer any of the pharmaceutical formulations with any of the foodstuffs disclosed herein. In another example, one can administer a microcapsule with any of the nutritional supplements disclosed herein. In yet another example, one can administer any of the pharmaceutical formulations with any of the delivery devices and nutritional supplement disclosed herein, and the like.

Dosage

When used in the above described methods or other treatments, or in the nutritional supplements, pharmaceutical formulations, delivery devices, or foodstuffs disclosed herein, an “effective amount” of one of the disclosed compounds can be employed in pure form or, where such forms exist, in pharmaceutically acceptable salt form and with or without a pharmaceutically acceptable excipient, carrier, or other additive.

The specific effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions may contain such amounts or submultiples thereof to make up the daily dose.

The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products. A typical daily dosage of the compounds disclosed herein used alone might range from about 10 to up to about 30 of 3000 micrograms or more per day, depending on the factors mentioned above.

Administration and Delivery

In one aspect, disclosed herein are uses of a delivery device to deliver a chromium compound to a subject. Further, disclosed are methods for delivering a chromium compound comprising one or more chromium atoms bonded to one or more fatty acid residues to a subject by administering to the subject any of the nutritional supplements, pharmaceutical formulations, delivery devices, and/or foodstuffs disclosed herein.

The compounds disclosed herein (including nutritional supplements, microcapsules, delivery devices, and pharmaceutical formulations) can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Production of Chromium Salts from CrCl₃

A solution of CrCl₃.6H₂O (7.98 g, 30 mmol) in 10 mL distilled H₂O was mixed with 4020FFA (Free Fatty Acid, prepared by hydrolysis of 4020EE, a product of Ocean Nutrition Canada, Mulgrave, NS) (10.2 g, 30 mmol) dissolved in 10 g hexane and refluxed together for 5 h (hours) under N₂ with vigorous stirring. The resulting mixture was separated in a separatory funnel, the aqueous phase was lyophilized and the lipid phase was evaporated to dryness. Lyophilized material was dissolved in 40 mL ethyl acetate, centrifuged and filtered to remove any unreacted CrCl₃, and evaporated. The product was freely soluble in hexane and was in a form of a very dark green liquid containing chromium at a concentration of 98 mg/g. The total mass recovery was 66%. The product can also be prepared in the absence of hexane.

### Example 2

### Production of Chromium Salts from Cr(OH)₃

A mixture of Cr(OH)₃ (8.49 g, 82.4 mmol), 3020 FFA (15 g, 45.5 mmol, prepared by hydrolysis of 3020EE, a product of Ocean Nutrition Canada, Mulgrave, NS), and 20 mL of 50% aqueous ethanol was reflexed under N₂ at 90° C. for 5 h. The resulting mixture was diluted with 60 mL hexane and centrifuged at 10,000 rpm for 30 minutes. The pellet was washed twice more with 60 mL of hexane, the supernatant filtered into a separatory funnel, and dried over sodium sulfate. Finally the drying agent was filtered off and the solution concentrated. The product was freely soluble in hexane and was in the form of a very dark green liquid containing chromium at a concentration of 8.9 mg/g. The total mass recovery was 15%.

According to Hein and Herzog (Chromium, Molybdenum, Tungsten, Uranium. In: Handbook of Preparative Inorganic Chemistry, Vol. 2. Brauer G., Academic Press, New York, pp. 1334-1401, 1965) chromium(III) hydroxide exists in a form of A-hydroxide-Cr(OH)₃.3H₂O and B-hydroxide Cr(OH)₃.3H₂O. A-hydroxide gives blue salts with dilute acids of the formula [Cr(H₂O)₆]X₃, B-hydroxide gives green salts with dilute acids of the formula [Cr(H₂O)₄X₂]X.2H₂O. The starting material was B-hydroxide since it was not soluble in acetic acid and the product was dark green. Although one would expect similar composition of the disclosed chromium complexes, lipophilicity, and stereochemical factors of long chain fatty acids are so significant that they can totally change the composition of these complexes and they may be very similar to the product prepared according to Example 1.

### Example 3

### Production of Chromium Salts from CrCl₂

Sodium salt of hydrolyzed fish oil was produced by combining 4020 FFA (34.0 g, 100 mmol) with NaOH (4.0 g, 100 mmol) dissolved in 50 mL of distilled H₂O. Next, 5 g CrCl₂ in 25 mL of distilled H₂O was added and the mixture refluxed under N₂ at 90° C. for 5 h. The mixture was allowed to cool briefly and was filtered to remove chunks of unreacted sodium-fish oil salt. The filtrate was separated in a funnel to remove most of the water and then the organic phase was dried over sodium sulfate. The drying agent was filtered off and the filtrate concentrated. The product was freely soluble in hexane and was a dark purple liquid containing chromium at a concentration of 55 mg/mL. The total mass recovery was 78%.

The reaction between CrCl₂ and FFA is believed to produce dinuclear complex resembling the following Formula II:

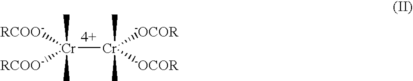

where R is the omega-3 fatty acid chain, e.g., from DHA.

### Example 4

### Structural Determination

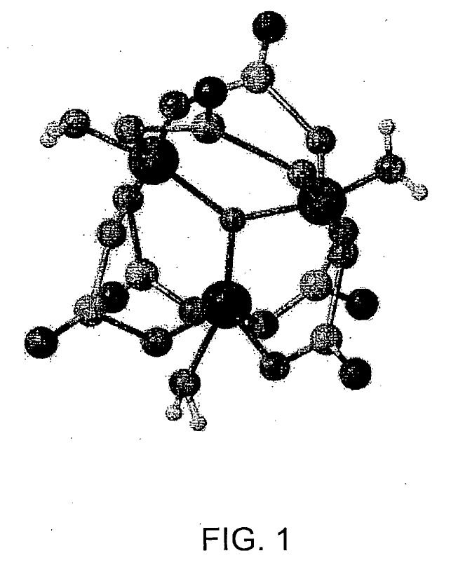

CrCl₃ is a red-violet crystalline scale with metallic luster. This material is not soluble and its solubility is facilitated by adding a trace of CrCl₂. The chromium chloride preparation was in the form of blue gray crystals that would indicate rather hexaaquachromium(III) chloride Cr(H₂O)₆Cl₃. They are readily soluble in alcohol and insoluble in acetone. Pentaaquachromium(III) chloride Cr(H₂O)₅Cl₃ is a bright green material readily soluble in water, alcohol and acetone. Octahedral d3 coordination is typical for Cr(III). It can be difficult to determine the structure of these complexes even if only one fatty acid chain was involved. Although the complexes are generally kinetically inert, the composition of the final products depends on physical parameters and environmental conditions. They can be in a form of tri-nuclear complex of a general formula:

[Cr₃(H₂O)₃(μ-OCOR)₆(μ₃-O)]⁺

where k is the omega-3 fatty acid chain, e.g., from DHA. The proposed structure of the complex is shown in FIG. 1.

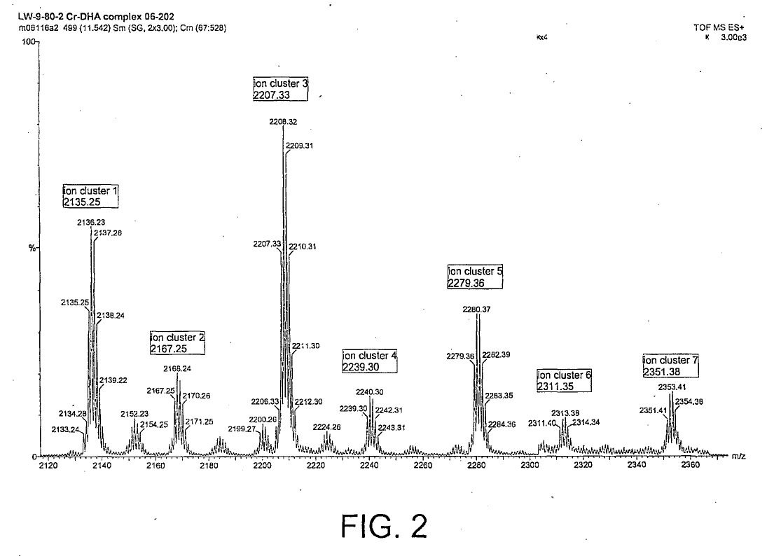

To analyze the structure of the chromium complexes, the DHA-Cr salt produced according to Example 1 was diluted with a mixture of tetrahydrofuran (THF) and methanol 90:10 and the solution was infused into an electrospray source for characterization. The source was optimized to minimize fragmentation and provide intact parent ion complexes. FIG. 2 is the spectrum that resulted from scanning the instrument. The spectrum showed several similar isotope patterns that resulted from chromium complexes due to the fact that they were centered at distinct m/z values. These complexes could be interpreted in such a way as to allow structural characterization of the Cr-DHA product based on the assumption that the product was a tri-nuclear chromium complex with a single bridging oxide ion and six DHA anionic bidentate ligands to form the core structure in addition to 3 labile outer ligands that under rapid exchange with solvent molecules. The assumptions about the structure came from the work of van den Bergen et al. “Electrospray mass spectrometric study of [M₃O(RCOO)₆L₃]⁺ cations (M=Cr, Fe; L=H₂O, MeOH, py.” Inorg Chem 32:3408-11, 1993, and lead to the generalized molecular formula of Cr₃O[RCOO]₆.[H₂O]₃.

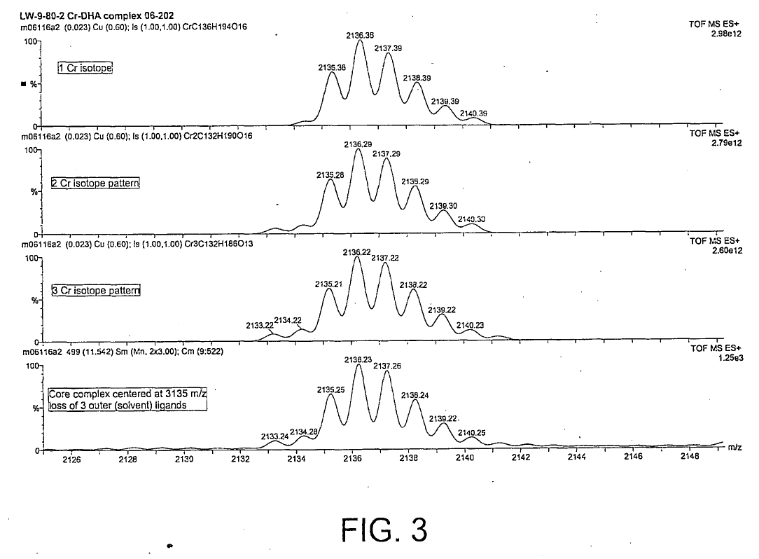

Each of the isotope patterns portrayed similar distributions that suggested that the number of chromium atoms was constant. The fact that a multinuclear chromium complex was present can be inferred from the isotope pattern. FIG. 3 compares the isotope cluster for core complex at 2135.2 m/z to three computationally derived clusters based on the proposed molecular formula and a select number of chromium atoms. The best, agreement occurs for the isotope pattern that was generated using three chromium atoms in the molecular formula. There would be small differences between the spectra based on the limitations of the instrument design and low signal to noise such that a completely unambiguous determination may not be possible. The difference between a di- and trinuclear complex was within the error associated with the spectral measurement. A mononuclear chromium complex was the least favored outcome indicated for this data. The mass variance between the observed and the calculated molecular weight value of the core complex ion (2135.25 m/z vs. 2135.2177 m/z expected) was due to the tuning and calibration of the instrument. The QTOF (Quadrapol-Time-of-Flight) mass spectrometer was operated at the limit of the sensitivity for these experiments and so 5 ppm mass accuracy was not obtained. Typical accuracy was still respectable at better than 20 ppm for these experiments. Reliable structural information can still be inferred from this data so long as constraints are placed on the expected outcomes in terms of the atoms expected from the molecular formula.

As previously discussed, a tri-nuclear chromium complex was postulated based on the work of van den Bergen. The ions in FIG. 2 were examined to help characterize the structure assuming a core component and facile ligand exchange with the THF and methanol solvent molecules. The core complex was believed to have the molecular formula of Cr₃O[C₂₂H₃₁O₂]₆, where the six fatty acid DHA bridging ligands bound strongly to the chromium skeleton. A further three labile axial ligands complete the structure. These ligands were believed to be water in the solid state according to the literature but were replaced by THF and methanol in this example. The ion clusters observed in FIG. 2 support this assignment. A description of the composition of the ion cluster assignment follows in Table 5.

TABLE 5
Molecular formula assignment of ion clusters observed from the mass
spectrum of a Cr-DHA complex dissolved in THF and methanol

| | | Expected center | |
|---|---|---|---|
| | Measured | mass of isotope | |
| | center mass | distribution | |
| Ion | of isotope | based on formula | |
| cluster | distribution | assignment | Formula Assignment |
| 1 | 2135.25 m/z | 2135.21 m/z | Core = Cr₃O[C₂₂H₃₁O₂]₆ |
| 2 | 2167.25 m/z | 2167.24 m/z | Core + CH₄O (methanol) |
| 3 | 2207.33 m/z | 2207.27 m/z | Core + C₄H₈O (THF) |
| 4 | 2239.90 m/z | 2239.40 m/z | Core + (THF) + (methanol) |
| 5 | 2279.36 m/z | 2279.33 m/z | Core + (THF)₂ |
| 6 | 2311.35 m/z | 2311.40 m/z | Core + (THF)₂ + (methanol) |
| 7 | 2351.41 m/z | 2351.38 m/z | Core + (THF)₃ |

The assignment of the molecular formula from Table 5 of the ion complexes was consistent with that for a tri-nuclear chromium carboxylic acid complex. Although accurate mass measurements for a single ion were not able to be obtained, the mass accuracy of all of the ion complexes measured dramatically increases the probability of a correct molecular formula determination for the Cr-DHA complex.

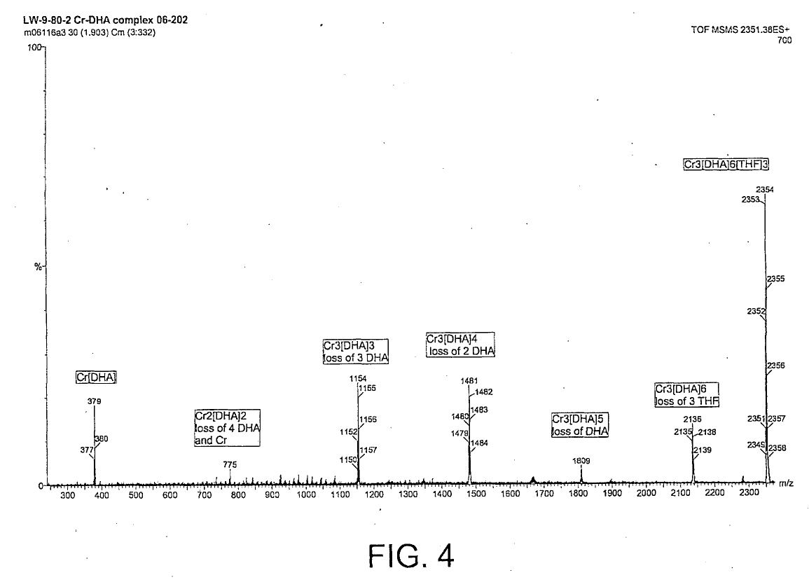

In order to further confirm the structural properties of the Cr-DHA complex the parent ion was fragmented using collision activated dissociation in this experiment the parent ion (2351.4 m/z) was introduced into a collision cell that was pressurized with inert argon gas. The ions were accelerated into this gas cloud with 120 V of energy. This causes the molecule to break apart or fragment into structurally diagnostic components. FIG. 4 was the spectrum that resulted from this experiment. The fragment ions were labeled in FIG. 4 and the data was consistent with earlier molecular formula assignment. The parent ion complex Cr₃O[C₈H₁₅O₂]₆[THF]₃⁺ was observed at 2351.4 m/z. Loss of three THF molecules followed by sequential loss of DHA ligands was observed from the parent ion complex. The low mass ion series can also be used to assign structural information. In addition, Cr-DI-IA was observed at 379.2 m/z.

To summarize, mass spectrometry indicated that a tri-nuclear Cr-DHA complex was formed with the general formula Cr₃O[C₂₂H₃₁O₂]₆[H₂O]₃. The water ligands were displaced by methanol and THF molecules during dilution prior to infusion into the mass spectrometer. The assignment of water ligands came from the van den Bergen paper. Previous experiments with caprylic acid confirmed that water was present as a ligand. However, the presence of water in the Cr-DHA structure was not confirmed and it can be the case that DHA ethylesters may provide a counter ligand as the solvating liquid. This may not be entirely unexpected as Cr-DHA is much more lipophilic compared with the shorter chain fatty acids studied.

Throughout this application, various publications are referenced. The disclosures of these publications in their entireties are hereby incorporated by reference into this application.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Example 5

### Biological Data

### Effect of Cr-Omega-3 Conjugate on Fasting Blood Glucose and Glucose Tolerance in Diabetic Mice

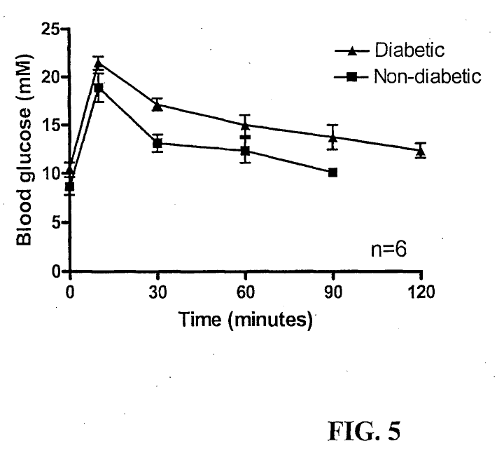

Mice (C57BL/6J) weighing 20-grams were fed a high fat, high sucrose diet with no added chromium (No. D01030101, Research Diets Inc., New Brunswick, N.J.). After two weeks, they were injected intraperitoneally (i.p.) with streptozotocin (in phosphate-buffered saline, PBS) at a dose of 35 mg/kg body weight on 3 consecutive days. The combination of the high fat/high sucrose diet and streptozotocin treatment produced an impaired glucose tolerance evident 7 days following the initial streptozotocin injection (FIG. 5). Glucose tolerance was measured in mice fasted overnight and then injected i.p. with 1 g glucose/kg body weight (0.25 mL of a 10% glucose solution in PBS). Blood glucose was measured using a glucometer (OneTouch Ultra, LifeScan Inc., Milpitas, Calif.) on a drop of blood obtained from the saphenous vein just prior to (0 time) and at 10, 30, 60, 90 and 120 minutes following injection.

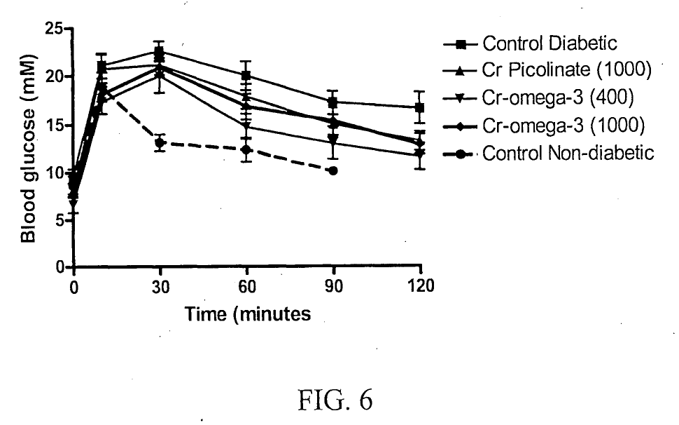

Animals were subsequently assigned to one of four different diet treatments, six animals per group. The diets were modified from the above high fat, high sucrose diet (Table 6). Each diet contained 1.5% fish oil concentrate (40:20 EPA/DHA ethyl ester, Ocean Nutrition Canada, Ltd., Mulgrave, NS). One diet had no added chromium (Control), whereas the other three diets contained either Cr-omega-3 conjugate (400 and 1000 g elemental Cr/kg diet) or chromium picolinate (1000 μg elemental Cr/kg diet). The animals were fed these diets for 4 weeks, after which time, fasting blood glucose and glucose tolerance was evaluated. The results showed that the dietary Cr provided either as Cr picolinate or Cr-omega-3 conjugate resulted in a reduction in fasting blood glucose compared to the level in the control group. Fast blood glucose levels were 9.6±1.8, 8.17±1.8, 6.65±1.8, and 7.68±0.7 mM, for the Control, Cr picolinate, Cr-n3 (400 μg Cr/kg diet), and Cr-n3 (1000 μg Cr/kg diet) groups, respectively. The mice on these diets tended to have improved glucose tolerance compared to the control (FIG. 6). Mouse diet without added chromium (D01030104, Research Diets Inc., New Brunswick, N.J.) was supplemented with fish oil concentrate (Ocean Nutrition Canada, Ltd., Mulgrave, NS), chromium picolinate or chromium omega-3 conjugate.

TABLE 6
Fish oil and Cr content of Control and test diets

| | Diets | | | |
|---|---|---|---|---|
| | | | Cr-Omega-3 | Cr-Omega-3 |
| | | Cr | 400 | 1000 |
| Additions (grams) | Control | Picolinate | (μg Cr/kg) | (μg Cr/kg) |
| Casein | 200 | 200 | 200 | 200 |
| L-cystine | 3 | 3 | 3 | 3 |
| Corn starch | 72.8 | 72.8 | 72.8 | 72.8 |
| Sucrose | 172.8 | 172.8 | 172.8 | 172.8 |
| Maltodextrin | 100 | 100 | 100 | 100 |
| fiber | 50 | 50 | 50 | 50 |
| Soybean Oil | 25 | 25 | 25 | 25 |
| Lard | 164.5 | 164.5 | 164.5 | 164.5 |
| Fish Oil (g/kg) | 13 | 13 | 13 | 13 |
| Mineral Mix S17902 | 10 | 10 | 10 | 10 |
| DiCalcium phosphate | 13 | 13 | 13 | 13 |
| Calcium carbonate | 5.5 | 5.5 | 5.5 | 5.5 |
| Potassium citrate | 16.5 | 16.5 | 16.5 | 16.5 |
| Vitamin mix V10001 | 10 | 10 | 10 | 10 |
| Choline bitartrate | 2 | 2 | 2 | 2 |
| Cr Picolinate | — | 0.0072 | — | — |
| Cr-omega-3 conjugate | — | — | 0.012 | 0.029 |
| Total (g) | 858.1 | 858.1 | 858.1 | 858.1 |
| Mg Chromium/kg | 0 | 1007 | 420 | 1014 |

1. A compound, comprising one or more chromium atoms bonded to one or more unsaturated fatty acid residues, wherein the unsaturated fatty acid residue is not derived solely from oleic acid.

2. The compound of claim 1, wherein the unsaturated fatty acid residue is derived from fish oil.

3. The compound of claim 1, wherein the unsaturated fatty acid residue comprises at least 20 carbon atoms.

4. The compound of claim 1, wherein the unsaturated fatty acid residue comprises at least one pair of methylene interrupted unsaturated bonds.

5. The compound of claim 1, wherein the unsaturated fatty acid residue is derived from an omega-3 fatty acid.

6. The compound of claim 1, wherein the unsaturated fatty acid residue is derived from a compound comprising the formula: 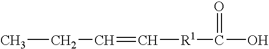
wherein R¹ is a C₃-C₄₀ alkyl or alkenyl group comprising at least one double bond.

7. The compound of claim 6, wherein R¹ has from 2 to 6 double bonds.

8. The compound of claim 1, wherein the unsaturated fatty acid residue is derived from linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof.

9. The compound of claim 1, wherein the unsaturated fatty acid residue is derived from eicosapentaenoic acid 20:5ω3 (EPA), docosahexaenoic acid 22:6ω3 (DHA), docosapentaenoic acid 22:5ω3 (DPA), or any mixture thereof.

10. The compound of claim 1, wherein the number of chromium atoms is from 1 to 3.

11. The compound of claim 1, wherein the number of chromium atoms is 3.

12. The compound of claim 1, further comprising one or more water molecules bonded to the chromium atom.

13. The compound of claim 1, further comprising a non-chromium atom bonded to one or more unsaturated fatty acid residues, wherein the non-chromium atom comprises a metal, transition metal, alkaline metal, an alkaline earth metal, rare earth metal, or metalloid.

14. The compound of claim 13, wherein the non-chromium compound is Zn, Mn, W, Mo, V, Nb Ta, Ga, La, Sb.

15. The compound of claim 1, wherein the compound comprises the formula:
[Cr₃(H₂O)₃(μ-unsaturated fatty acid residue)₆(μ₃-O)]⁺.

16. The compound of claim 1, wherein the compound comprises the formula:
[Cr₃(H₂O)₃(μ-O₂C—R¹—CH═CH—CH₂—CH₃)₆(μ₃-O)]⁺
wherein R¹ is a C₃-C₄₀ alkenyl group comprising at least one double bond.

17. The compound of claim 16, wherein R¹ has from 2 to 6 double bonds.

18. The compound of claim 15, wherein the unsaturated fatty acid residue is derived from linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof.

19. The compound of claim 15, wherein the unsaturated fatty acid residue is derived from eicosapentaenoic acid 20:5ω3 (EPA), docosahexaenoic acid 22:6ω3 (DHA), docosapentaenoic acid 22:5ω3 (DPA), or any mixture thereof.

20. The compound of claim 1, wherein the chromium is chromium(III).

21. (canceled)

22. (canceled)

23. (canceled)

24. The compound of claim 1, wherein the compound is a liquid.

25. A method for preparing a compound, comprising reacting a chromium compound and one or more unsaturated fatty acids or the salt or ester thereof, wherein the unsaturated fatty acid or the salt or ester thereof is not solely oleic acid.

26. The method of claim 25, wherein the chromium compound is in the form of a hydrate.

27. The method of claim 25, wherein the chromium compound is a chromium(III) compound.

28. The method of claim 25, wherein the chromium(D) compound is CrCl₃ or a hydrate thereof.

29. The method of claim 25, wherein the chromium(III) compound is Cr(OH)₃ or a hydrate thereof.

30. The method of claim 25, wherein the chromium compound is a chromium(I) compound.

31. The method of claim 30, wherein the chromium(II) compound is CrCl₂, or a hydrate thereof.

32. The method of claim 25, wherein the chromium compound comprises Cr(SO₄)₂, CrCl₂.4H₂O, CrS, CrO, CrBr₃.6H₂O, CrF₃, CrF₃.4H₂O, CrCl₃, KCr(SO₄)₂.12H₂O, Cr₂(SO₄)₃.xH₂O, Cr₂S₃, [Cr(H₂O)₄Cl₂]Cl.2H₂O, Cr(H₂O)₆Cl₃, Cr₂O₃, Cr₂O₃.xH₂O, CrPO₄.4H₂O, where x is an integer from 1 to 28, or any mixture thereof.

33. The method of claim 25, wherein the reacting step is performed in one or more aqueous solvents.

34. The method of claim 25, wherein the reacting step does not involve a reducing agent.

35. The method of claim 25, wherein the reacting step is performed at an elevated temperature.

36. The method of claim 25, wherein the unsaturated fatty acid is derived from fish oil.

37. The method of claim 25, wherein the unsaturated fatty acid comprises at least 20 carbon atoms.

38. The method of claim 25, wherein the unsaturated fatty acid comprises at least one pair of methylene interrupted unsaturated bonds.

39. The method of claim 25, wherein the unsaturated fatty acid is an omega-3 fatty acid.

40. The method of claim 25, wherein the unsaturated fatty acid comprises the formula: 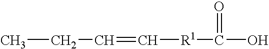
wherein R¹ is a C₃-C₄₀ alkyl or alkenyl group comprising at least one double bond.

41. The method of claim 40, wherein R¹ has from 2 to 6 double bonds.

42. The method of claim 25, wherein the unsaturated fatty acid is linoleic acid, linolenic acid, gamma-linolenic acid, arachidonic acid, mead acid, stearidonic acid, alpha-eleostearic acid, eleostearic acid, pinolenic acid, docosadienic acid, docosatetraenoic acid, octadecadienoic acid, octadecatrienoic acid, eicosatetraenoic acid, or any combination thereof.

43. The method of claim 25, wherein the unsaturated fatty acid comprises eicosapentaenoic acid 20:5ω3 (EPA), docosahexaenoic acid 22:6ω3 (DHA), docosapentaenoic acid 22:5ω3 (DPA), or any mixture thereof.

44. The method of claim 25, further comprising reacting the unsaturated fatty acid with a non-chromium compound, wherein the non-chromium compound comprises a metal, transition metal, an alkaline metal, alkaline earth metal, rare earth metal, or metalloid.

45. The method of claim 44, wherein the non-chromium compound is Zn, Mn, W, Mo, V, Nb Ta, Ga, La, or Sb.

46. A compound prepared by the method of claim 25.

47. A nutritional supplement comprising a chromium compound comprising one or more chromium atoms bonded to one or more fatty acid residues.

48. (canceled)

49. The nutritional supplement of claim 47, comprising from about 10 to about 3000 micrograms of chromium.

50. The nutritional supplement of claim 47, comprising from about 50 to about 200 micrograms of chromium.

51. The nutritional supplement of claim 47, wherein the supplement is in the form of a tablet, gel-cap, capsule, liquid, or syrup.

52. A delivery device comprising a chromium compound comprising one or more chromium atoms bonded to one or more fatty acid residues.

53. The delivery device of claim 52, wherein the device comprises a microcapsule, a microsphere, a nanosphere or nanoparticle, a liposome, a noisome, a nanoerythrosome, a solid-liquid nanoparticle, a leuprolide, a gel, a gel capsule, a tablet, a lotion, a cream, a spray, an emulsion, or a powder.

54. The delivery device of claim 52, wherein the device comprises a microcapsule, wherein the microcapsule comprises an agglomeration of primary microcapsules, each individual primary microcapsule having a primary shell and the agglomeration being encapsulated by an outer shell, wherein a chromium compound comprising one or more chromium atoms bonded to one or more fatty acid residues is encapsulated in the primary microcapsule.

55. The delivery device of claim 54, wherein the primary shell and the outer shell comprises gelatin type A, gelatin type B, polyphosphate, gum arabic, alginate, chitosan, carrageenan, pectin, starch, modified starch, alfa-lactalbumin, beta-lactoglobumin, ovalbumin, polysorbiton, maltodextrins, cyclodextrins, cellulose, methyl cellulose, ethyl cellulose, hydropropylmethylcellulose, carboxymethylcellulose, milk protein, whey protein, soy protein, canola protein, albumin, chitin, polylactide, poly-lactide-co-glycolide, polylysine, kosher gelatin, non-kosher gelatin, Halal gelatin, non-Halal gelatin, or a mixture thereof.

56. The delivery device of claim 54, wherein the primary shell and the outer shell comprises gelatine type A having a Bloom strength of from 0 to 350.

57. The delivery device of claim 54, wherein the primary shell and the outer shell comprises a zero bloom fish gelatin.

58. The delivery device of claim 54, further comprising an additional shell surrounding the outer shell, wherein at least one of the primary, outer, and additional shells comprise a complex coacervate.

59. A foodstuff comprising a chromium compound comprising one or more chromium atoms bonded to one or more fatty acid residues.

60. The foodstuff of claim 59, wherein the foodstuff is a baked good, a pasta, a meat product, a frozen dairy product, a milk product, a cheese product, an egg product, a condiment, a soup mix, a snack food, a nut product, a plant protein product, a hard candy, a soft candy, a poultry product, a processed fruit juice, a granulated sugar, a sauce, a gravy, a syrup, a nutritional bar, a beverage, a dry beverage powder, a jam or jelly, a fish product, or pet companion food.

61. The foodstuff of claim 59, wherein the foodstuff is bread, tortillas, cereal, sausage, chicken, ice cream, yoghurt, milk, salad dressing, rice bran, fruit juice, a dry beverage powder, rolls, cookies, crackers, fruit pies, or cakes.

62. (canceled)

63. (canceled)

64. A method improving insulin sensitivity in a subject, comprising the step of administering an effective amount of a chromium compound comprising one or more chromium atoms bonded to one or more fatty acid residues, a nutritional supplement comprising the chromium compound, a delivery device comprising the chromium compound, or a foodstuff comprising the chromium compound to the subject.

65. A method of reducing hyperglycemia in a subject, comprising the step of administering an effective amount of a chromium compound comprising one or more chromium atoms bonded to one or more fatty acid residues, a nutritional supplement comprising the chromium compound, a delivery device comprising the chromium compound, or a foodstuff comprising the chromium compound to the subject.

66. (canceled)

67. (canceled)

68. (canceled)

69. A method of treating or preventing diabetes in a subject, comprising the step of administering an effective amount of a chromium compound comprising one or more chromium atoms bonded to one or more fatty acid residues, a nutritional supplement comprising the chromium compound, a delivery device comprising the chromium compound, or a foodstuff comprising the chromium compound to the subject.

70. The method of claim 69, wherein the treating diabetes comprises reducing the blood glucose level in the subject.

71. (canceled)

72. A pharmaceutical formulation comprising a chromium compound comprising one or more chromium atoms bonded to one or more fatty acid residues and a pharmaceutically acceptable carrier.

73. A method for lowering triglycerides in a subject comprising administering the composition of claim 1, wherein the composition comprises an omega 3 fatty acid, to the subject, wherein the subject is desirous of lowering its triglyceride level.

74. A method for lowering depression in a subject comprising administering the composition of claim 1, wherein the composition comprises an omega 3 fatty acid, to the subject, wherein the subject is desirous of lowering its depression level.

75. A method for lowering inflammation in a subject comprising administering the composition of claim 1, wherein the composition comprises an omega 3 fatty acid, to the subject, wherein the subject is desirous of lowering its inflammation level.

76. A method for lowering blood pressure in a subject comprising administering the composition of claim 1, wherein the composition comprises an omega 3 fatty acid, to the subject, wherein the subject is desirous of lowering its blood pressure level.

77. A method for lowering arrhythmias in a subject comprising administering the composition of claim 1, wherein the composition comprises an omega 3 fatty acid, to the subject, wherein the subject is desirous of lowering its arrhythmias.

78. A method for increasing visual acuity in a subject comprising administering the composition of claim 1, wherein the composition comprises an omega 3 fatty acid to the subject, wherein it is desired that the subject increases its visual acuity.

79. A method for increasing cognitive development in a subject comprising administering the composition of claim 1, wherein the composition comprises an omega 3 fatty acid to the subject, wherein it is desired that the subject increases its cognitive development.
